SEQUENCE LISTING (36 sequences)

SEQ ID NO: 1 - protein, 7 aa, Artificial Sequence
  Description of Artificial Sequence Synthetic peptide
YGFVWGE

SEQ ID NO: 2 - protein, 7 aa, Artificial Sequence
  Description of Artificial Sequence Synthetic peptide
YGFVWGQ

SEQ ID NO: 3 - protein, 7 aa, Artificial Sequence
  Description of Artificial Sequence Synthetic peptide
YGFLWGQ

SEQ ID NO: 4 - protein, 7 aa, Artificial Sequence
  Description of Artificial Sequence Synthetic peptide
YGFLWGE

SEQ ID NO: 5 - protein, 7 aa, Artificial Sequence
  Description of Artificial Sequence Synthetic peptide
YGFAWGQ

SEQ ID NO: 6 - protein, 7 aa, Artificial Sequence
  Description of Artificial Sequence Synthetic peptide
YGFAWGE

SEQ ID NO: 7 - protein, 7 aa, Artificial Sequence
  Description of Artificial Sequence Synthetic peptide
YGFIWGQ

SEQ ID NO: 8 - protein, 7 aa, Artificial Sequence
  Description of Artificial Sequence Synthetic peptide
YGFIWGE

SEQ ID NO: 9 - protein, 9 aa, Artificial Sequence
  Description of Artificial Sequence Synthetic peptide
CYGFVWGEC

SEQ ID NO: 10 - protein, 9 aa, Artificial Sequence
  Description of Artificial Sequence Synthetic peptide
CYGFVWGQC

SEQ ID NO: 11 - protein, 9 aa, Artificial Sequence
  Description of Artificial Sequence Synthetic peptide
CYGFLWGQC

SEQ ID NO: 12 - protein, 9 aa, Artificial Sequence
  Description of Artificial Sequence Synthetic peptide
CYGFLWGEC

SEQ ID NO: 13 - protein, 9 aa, Artificial Sequence
  Description of Artificial Sequence Synthetic peptide
CYGFAWGQC

SEQ ID NO: 14 - protein, 9 aa, Artificial Sequence
  Description of Artificial Sequence Synthetic peptide
CYGFAWGEC

SEQ ID NO: 15 - protein, 9 aa, Artificial Sequence
  Description of Artificial Sequence Synthetic peptide
CYGFIWGQC

SEQ ID NO: 16 - protein, 9 aa, Artificial Sequence
  Description of Artificial Sequence Synthetic peptide
CYGFIWGEC

SEQ ID NO: 17 - protein, 9 aa, Artificial Sequence
  Description of Artificial Sequence Synthetic peptide
  MOD_RES (1): D-Ala
AYGFVWGEE

SEQ ID NO: 18 - protein, 9 aa, Artificial Sequence
  Description of Artificial Sequence Synthetic peptide
  MOD_RES (1): D-Ala
AYGFVWGQE

SEQ ID NO: 19 - protein, 9 aa, Artificial Sequence
  Description of Artificial Sequence Synthetic peptide
  MOD_RES (1): D-Ala
AYGFLWGQE

SEQ ID NO: 20 - protein, 9 aa, Artificial Sequence
  Description of Artificial Sequence Synthetic peptide
  MOD_RES (1): D-Ala
AYGFLWGEE

SEQ ID NO: 21 - protein, 9 aa, Artificial Sequence
  Description of Artificial Sequence Synthetic peptide
  MOD_RES (1): D-Ala
AYGFAWGQE

SEQ ID NO: 22 - protein, 9 aa, Artificial Sequence
  Description of Artificial Sequence Synthetic peptide
  MOD_RES (1): D-Ala
AYGFAWGEE

SEQ ID NO: 23 - protein, 9 aa, Artificial Sequence
  Description of Artificial Sequence Synthetic peptide
  MOD_RES (1): D-Ala
AYGFIWGQE

SEQ ID NO: 24 - protein, 9 aa, Artificial Sequence
  Description of Artificial Sequence Synthetic peptide
  MOD_RES (1): D-Ala
AYGFIWGEE

SEQ ID NO: 25 - protein, 9 aa, Artificial Sequence
  Description of Artificial Sequence Synthetic peptide
  MOD_RES (1): Variable amino acid that may or may not be present
  MOD_RES (5): Ala, Val, Leu or Ile
  MOD_RES (9): Variable amino acid that may or may not be present
  see specification as filed for detailed description of substitutions and preferred embodiments
XYGFXWGZX

SEQ ID NO: 26 - protein, 7 aa, Artificial Sequence
  Description of Artificial Sequence Synthetic peptide
  MOD_RES (4): Ala, Val, Leu or Ile
  see specification as filed for detailed description of substitutions and preferred embodiments
YGFXWGZ

SEQ ID NO: 27 - protein, 9 aa, Artificial Sequence
  Description of Artificial Sequence Synthetic peptide
  MOD_RES (5): Ala, Val, Leu or Ile
CYGFXWGZC

SEQ ID NO: 28 - protein, 9 aa, Artificial Sequence
  Description of Artificial Sequence Synthetic peptide
  MOD_RES (1): Variable amino acid
  MOD_RES (5): Ala, Val, Leu or Ile
  MOD_RES (9): Variable amino acid
  see specification as filed for detailed description of substitutions and preferred embodiments
XYGFXWGZX

SEQ ID NO: 29 - protein, 16 aa, Artificial Sequence
  Description of Artificial Sequence Synthetic peptide
RQIKIWFQNRRMKWKK

SEQ ID NO: 30 - protein, 7 aa, Artificial Sequence
  Description of Artificial Sequence Synthetic peptide
KLAKLAK

SEQ ID NO: 31 - DNA, 29 bp, Artificial Sequence
  Description of Artificial Sequence Synthetic primer
gcaagctgataaaccgatacaattaaagg

SEQ ID NO: 32 - DNA, 24 bp, Artificial Sequence
  Description of Artificial Sequence Synthetic primer
gccctcatagttagcgtaacgatc

SEQ ID NO: 33 - protein, 4 aa, Artificial Sequence
  Description of Artificial Sequence Synthetic peptide
ADGA

SEQ ID NO: 34 - protein, 13 aa, Artificial Sequence
  Description of Artificial Sequence Synthetic peptide
ADGACYGFVWGEC

SEQ ID NO: 35 - protein, 13 aa, Artificial Sequence
  Description of Artificial Sequence Synthetic peptide
ADGACYGFLWGQC

SEQ ID NO: 36 - protein, 13 aa, Artificial Sequence
  Description of Artificial Sequence Synthetic peptide
ADGACWEGGLYFC

(19) United States
(12) Patent Application Publication
(10) Pub. No.: US 2007/0258899 A1
(43) Pub. Date: Nov. 8, 2007

(54) Diagnostic and therapeutic agents

(75) Inventors: Seija Taube, Helsinki (FI); Mathias Bergman, Ostersundom (FI); Hannu Perakyla, Helsinki (FI); Hannu Elo, Helsinki (FI)

Correspondence Address: BUCHANAN, INGERSOLL & ROONEY PC, POST OFFICE BOX 1404, ALEXANDRIA, VA, 22313-1404, US

(73) Assignee: KARYON-CTT LTD, Helsinki (FI)

(21) Appl. No.: 11/730,290

(22) Filed: Mar. 30, 2007

Related U.S. Application Data
(60) Provisional application No. 60/787,519, filed on Mar. 31, 2006.

(51) Int. Cl. A61K 49/00; A61K 38/08; A61P 35/00; C07K 7/64; C07K 7/06; A61K 38/12

(52) U.S. Cl. 424/9.1; 514/15; 514/16; 514/9; 530/317; 530/328; 530/329

(57) ABSTRACT

Tumor targeting units are disclosed which have a peptide sequence C_{y}—Y—G-F—X—W-G-Z-C_{yy} (SEQ ID NO: 25), or a pharmaceutically or physiologically acceptable salt thereof. Tumor targeting agents are also disclosed having at least one targeting unit, directly or indirectly coupled to at least one effector unit. Diagnostic or pharmaceutical compositions having at least one targeting unit or at least one targeting agent, and targeting units or targeting agents for the preparation of a medicament for the treatment of cancer related diseases (including cancer), especially for the treatment of colon/colorectal cancer or its metastases are also disclosed.

### FIELD OF THE INVENTION

The present invention relates to targeting agents, especially to tumor targeting agents, such as colon/colorectal primary tumor and metastases targeting agents, comprising at least one targeting unit and at least one effector unit, as well as to tumor targeting units and motifs, such as colon/colorectal primary tumor and metastases targeting units and motifs. Further, the present invention concerns pharmaceutical and diagnostic compositions comprising such targeting agents or targeting units, and the use of such targeting agents and targeting units as pharmaceuticals or as diagnostic tools. The invention further relates to the use of such targeting agents and targeting units for the preparation of pharmaceutical or diagnostic compositions. Furthermore, the invention relates to kits for diagnosing or treating cancer, such as colon/colorectal primary tumor and metastases.

### BACKGROUND OF THE INVENTION

Malignant tumors are among the greatest health problems of man as well as animals, being one of the most common causes of death, also among young individuals. Available methods of treatment of cancer are quite limited, despite intensive research efforts during several decades. Although curative treatment, usually surgery in combination with chemotherapy and/or radiotherapy, is sometimes possible, malignant tumors still require a huge number of lives every year. In fact, curative treatment is rarely accomplished if the disease is not diagnosed early. In addition, certain tumor types can rarely, if ever, be cured.

There are various reasons for this very undesirable situation, the most important one clearly being the fact that most treatment schedules, except surgery, lack sufficient selectivity. Chemotherapeutic agents commonly used do not act on the malignant cells of the tumors alone but are highly toxic to other cells as well, especially to rapidly dividing cell types, such as hematopoietic and epithelial cells, resulting in highly undesirable side effects. The same applies to radiotherapy.

In addition, two major problems plague the non-surgical treatment of malignant solid tumors. Physiological barriers within tumors impede the delivery of therapeutics at effective concentrations to all cancer cells, and acquired drug resistance resulting from genetic and epigenetic mechanisms reduces the effectiveness of available drugs.

Also in the diagnosis of cancer and of metastases, including the follow-up of patients and the study of the effects of treatment on tumors and metastases, reliable, sensitive and more selective methods and agents would be a great advantage. All methods currently in use, such as nuclear magnetic resonance imaging, X-ray methods, histological staining methods still lack agents that are capable of targeting an entity for detection specifically or selectively to tumor tissues, metastases or tumor cells and/or to tumor endothelium.

According to The National Cancer Institute colorectal cancer is the third most common cancer and the third leading cause of cancer-related mortality in the United States. Over the past decade, colorectal cancer incidence and mortality rates have modestly decreased or remained level. Until age 50, men and women have similar incidence and mortality rates; after age 50, men are more vulnerable.

The prognosis of patients with colon cancer is clearly related to the degree of penetration of the tumor through the bowel wall, the presence or absence of nodal involvement, and the presence or absence of distant metastases. These three characteristics form the basis for all staging systems developed for this disease. Bowel obstruction and bowel perforation are indicators of poor prognosis.

See, e.g., the U.S. National Institutes of Health National Cancer Institute web site. Surgery is the treatment of choice for colorectal cancer. Treatment depends on the stage of the disease and the overall health of the patient. Radical bowel resection, also called partial colectomy and hemicolectomy, is used to treat 80-90% of colorectal cancer patients. Chemotherapy and radiation therapy may be used as adjuvant treatment.

Chemotherapy is a systemic treatment that often uses a combination of drugs to slow tumor growth and destroy cancer cells. It is often used as a first-line treatment for metastatic colorectal cancer. A combination of chemotherapy drugs (5-fluorouracil [5-FU], leucovorin, and irinotecan), administered intravenously, is standard treatment for metastatic colorectal cancer. Side effects include diarrhea, mucositis, neutropenia and alopecia. Newer combinations of chemotherapy drugs, such as FOLFOX (5-fluorouracil [5-FU], leucovorin, and oxaliplatin and FOFIRI (5-fluorouracil, leucovorin, and irinotecan may be used to prevent recurrence following surgery or to shrink the tumor prior to surgery.

In addition to chemotherapy drugs, blocking agents, e.g. cetuximab, (an anti-EGF mAb) may also be used to treat metastatic colorectal cancer. These drugs prevent cancer cell receptors from receiving factors (e.g., epidermal growth factor) that cause cell growth, cell division, and additional metastasis. Blocking agents target specific cells so they usually do not cause systemic side effects. Side effects of these drugs include allergic reactions.

An example of antiangiogenic drugs is bevacizumab (an anti-VEGF mAb), which may also be used to treat advanced colorectal cancer. This medication prevents new blood vessels, which are necessary for tumor growth, from forming. It does not affect normal tissues that already have an established blood supply. Side effects include blood clots and high blood pressure.

Immunotherapy, or biological therapy, attempts to stimulate the immune system to fight disease and protect the body from side effects of chemotherapy. Immunotherapy agents that may be used to treat colorectal cancer include bacilli Calmette-Guerin (BCG) and levamisole. Immunotherapy may cause flu-like side effects such as chills, diarrhea, fever, anorexia, muscle aches and weakness, nausea and vomiting.

Monoclonal antibodies specific to tumor cells have shown clinical promise as targeted agents for the treatment of e.g. lung cancer. There are some major limitations in antibody-targeted therapy based on two facts: the large size of the monoclonal antibodies and non-specific uptake of the antibody molecules by the liver and the reticuloendothelial system. The large size results in poor tumor penetration of antibody pharmaceuticals and causes often immune response, whereas non-specific uptake by the liver and the reticuloendothelial system results in dose-limiting toxicity to the liver and bone marrow. Another, hazardous disadvantage with the antibodies is their incorrect glycosylation when produced in cell culture.

Targeting peptides are an excellent alternative for targeted treatment of human cancers, and due to relatively small size they may overcome some of the problems with antibody targeting. Advantages of peptides are: Greater stability—peptides can be stored at room temperature for weeks; lower manufacturing costs (synthetic production versus recombinant production); rapid pharmacokinetics; excretion route that can be modified; and higher activity per mass of final targeting agent.

There are numerous publications disclosing peptides homing to different cell and tissue types. Some of these are claimed to be useful as cancer targeting peptides. Among the earliest identified homing peptides described are the integrin and NGR-receptor targeting peptides described by Ruoslahti et al., in e.g., U.S. Pat. No. 6,180,084.

International Patent publication WO 02/057299 describes a peptide suggested to inhibit cancer cell proliferation by binding to VEGF-R3.

No publications disclosing peptides selectively targeting colon cancer cells have been identified. Thus, there is a need for targeting agents useful in diagnosis and therapy of colon cancer.

### BRIEF DESCRIPTION OF THE INVENTION

The present invention relates to tumor targeting units, targeting to colon primary tumor and metastases, having a peptide sequence C_{y}—Y-G-F—X—W-G-Z-C_{yy} (SEQ ID NO: 25) or a pharmaceutically or diagnostically or physiologically acceptable salt or derivative thereof, wherein Y is tyrosine, or a structural or functional analogue thereof; G is glycine, or a structural or functional analogue thereof; F is phenylalanine, or a structural or functional analogue thereof; X is alanine, valine, leucine or isoleucine, or a structural or functional analogue thereof; W is tryptophan, or a structural or functional analogue thereof; Z is glutamine or glutamic acid, or a structural or functional analogue thereof; and C_{y} and C_{yy} are optional entities forming a cyclic structure. The targeting units of the present invention may be linear or cyclic or form part of a cyclic structure.

The invention further relates to tumor targeting agents comprising at least one targeting unit according to the present invention, directly or indirectly coupled to at least one effector unit. Preferably the effector unit is a directly or indirectly detectable substance or a therapeutic substance.

The invention further relates to tumor targeting agents further comprising optional units such as solubility enhancing units, preferably aqueous enhancing units.

The present invention further relates to diagnostic or pharmaceutical compositions comprising at least one targeting unit or at least one targeting agent according to the present invention, and to the use of targeting units or targeting agents according to the present invention for the preparation of a medicament for the treatment or diagnosis of cancer or cancer related diseases, especially for the treatment of colon cancer or its metastases.

The present invention further relates to methods for treating or diagnosing cancer or cancer related diseases by providing to a patient in need thereof a diagnostically or therapeutically effective amount of a pharmaceutical composition according to the present invention for diagnosing or treating colon cancer or its metastases.

### BRIEF DESCRIPTION OF THE DRAWINGS

In the following the invention will be described in greater detail by means of preferred embodiments with reference to the attached drawings, in which

FIG. 1A shows the binding of cancer cell lines HCT-15 (A), HCT-15-LM1 (B), HSC-3 (C) and C8161T (D), and control cell lines, mouse fibroblast cell line NIH3T3 (E) and murine endothelial cell line SVEC4-10 (F) to the immobilized targeting agent MJ012. Y-axis represents viable count.

FIG. 1B shows the binding of cancer cell lines HCT-15 (A), HCT-15-LM1 (B), HSC-3 (C) and C8161T (D), and, control cell lines, mouse fibroblast cell line NIH3T3 (E) and murine endothelial cell line SVEC4-10 (F) to the immobilized targeting agent MJ013. Y-axis represents viable count.

FIG. 2A shows the in vivo biodistribution of the targeting agent MJ017 after injection into the tail vein of tumor-bearing athymic mice. A=tumor, B=heart, C=lung, D=liver, E=spleen, F=small intestine and G=brain. Y-axis represents the europium content in a tissue as compared to the europium content in a muscle.

FIG. 2B shows the in vivo biodistribution of the targeting agent MJ018 after injection into the tail vein of tumor-bearing athymic mice. A=tumor, B=heart, C=lung, D=liver, E=spleen, F=small intestine and G=brain. Y-axis represents the europium content in a tissue as compared to the europium content in a muscle.

FIG. 3 shows the results of a cytotoxicity assay as viable count vs. time. LoVo cells were treated with Cu(SAO)₂ (A), DMSO (B), 138 pg/ml HP203 (C) or 5 μg/ml HP203 (D).

### DETAILED DESCRIPTION OF THE INVENTION

It is an object of the present invention to provide novel tumor targeting agents that comprise at least one targeting unit and, optionally, at least one effector unit. In an important embodiment, the invention provides targeting units comprising at least one motif capable of targeting solid tumors of the colon. As a specific embodiment, the present invention provides tumor targeting motifs and units that specifically target colon primary tumor cells and metastases.

The targeting units according to the present invention, optionally coupled to at least one effector unit, are therapeutically and diagnostically useful, especially in the treatment and diagnosis of cancer, including metastases, preferably tumors and metastases of the colon. Furthermore the targeting agents according to the present invention are useful for cell removal, selection, sorting and enrichment.

It is a second object of this invention to provide pharmaceutical and diagnostic compositions comprising at least one targeting agent or at least one targeting unit comprising at least one motif according to the present invention. Such compositions may be used to destroy tumors or hinder their growth, or for the diagnosis of cancer.

Preferred pharmaceutical and diagnostic compositions comprise at least one targeting agent or at least one targeting unit comprising at least one motif according to the present invention and an optional unit which is an aqueous solubility-enhancing unit.

As early diagnosis of metastases is very important for successful treatment of cancer, an important use of the targeting units and targeting agents of this invention is in early diagnosis of tumor metastases.

A third object of the present invention is to provide novel diagnostic and therapeutic methods and kits for the treatment and/or diagnosis of cancer, preferably cancer of the colon, including metastases.

The targeting units of this invention may be used as such or coupled to at least one effector unit.

For the purpose of this invention, the term “cancer” is used herein in its broadest sense, and includes any disease or condition involving transformed or malignant cells. In the art, cancers are classified into five major categories, according to their tissue origin (histological type): Carcinomas, sarcomas, myelomas, and lymphomas, which are solid tumor type cancers, and leukemias, which are “liquid cancers”. The term cancer, as used in the present invention, is intended to primarily include all types of diseases characterized by solid tumors, including disease states where there is no detectable solid tumor or where malignant or transformed cells, “cancer cells”, appear as diffuse infiltrates or sporadically among other cells in healthy tissue. The term “colon cancer” is herein intended to include both colon cancer and colorectal cancer.

The terms “amino acid” and “amino alcohol” are to be interpreted herein to include also diamino, triamino, oligoamino and polyamino acids and alcohols; dicarboxyl, tricarboxyl, oligocarboxyl and polycarboxyl amino acids; dihydroxyl, trihydroxyl, oligohydroxyl and polyhydroxyl amino alcohols; and analogous compounds comprising more than one carboxyl group or hydroxyl group and one or more amino groups. Any amino acid referred to in the present application is intended to include all isomers thereof, such as optical and geometrical isomers.

By the term “peptide” is meant, according to established terminology, a chain of amino acids (peptide units) linked together by peptide bonds to form an amino acid chain. Peptides may be linear or cyclic, and comprise branches, as described below. For the purposes of the present invention, also compounds comprising one or more D-amino acids, beta-amino acids and/or other unnatural amino acids (e.g. amino acids with unnatural side chains) are included in the term “peptide”. For the purposes of the present invention, the term “peptide” is intended to include peptidyl analogues comprising modified amino acids. Such modifications may for example comprise the introduction or presence of a substituent; the introduction or presence of an “extra” functional group such as an amino, hydrazino, carboxyl, formyl (aldehyde) or keto group, or another moiety; and the absence or removal of a functional group or other moiety. The term also includes analogues modified in the amino and/or carboxy termini, such as peptide amides and N-substituted amides, peptide hydrazides, N-substituted hydrazides, peptide esters, and their like, and peptides that do not comprise the amino-terminal —NH₂ group or that comprise e.g. a modified amino-terminal amino group or an imino or a hydrazino group instead of the amino-terminal amino group, and peptides that do not comprise the carboxy-terminal carboxyl group or comprise a modified group instead of it, and so on.

Some examples of possible reaction types that can be used to modify peptides, forming “peptidyl analogues”, are e.g., condensation and nucleophilic addition reactions as well as esterification, amide formation, formation of substituted amides, N-alkylation, formation of hydrazides, and salt formation. Salt formation may be the formation of any type of salt, such as alkali or other metal salt, ammonium salt, salts with organic bases, acid addition salts etc. Peptidyl analogues may be synthesized either from the corresponding peptides or directly (via other routes).

The expression “structural or functional analogues” of the peptides of the invention is used to encompass compounds that do not consist of amino acids or not of amino acids alone, or some or all of whose building blocks are modified amino acids. Different types of building blocks can be used for this purpose, as is well appreciated by those skilled in the art. The function of these compounds in biological systems is essentially similar to the function of the peptides. The resemblance between these compounds and the original peptides is thus based on structural and functional similarities. Such compounds are called peptidomimetic analogues, as they mimic the function, conformation and/or structure of the original peptides and, for the purposes of the present invention, they are included in the term “peptide”.

A functional analogue of a peptide according to the present invention is characterized by a binding ability with respect to the binding to tumors, tumor tissue, tumor cells or tumor endothelium which is essentially similar to that of the peptides they resemble. For example, compounds like benzolactam or piperazine containing analogues based on the structure of the peptide bond comprising structures of the original amino acids can be used as amino acid analogues (Adams et al. 1999, J. Immunol. Methods, 231: 249-260; Nakanishi and Kahn, 1996, In: The practice of medical chemistry, pp. 571-590, Academic Press; Houghten et al., 1999, J. Med. Chem., 42: 3743-3778; Nargund et al., 1998, J. Med. Chem., 41: 3103-3127).

A large variety of different types of peptidomimetic substances have been reported in the scientific and patent literature and are well known to those skilled in the art. Peptidomimetic substances (analogues) may comprise for example one or more of the following structural components: reduced amides, hydroxyethylene and/or hydroxyethylamine isosteres, N-methyl amino acids, urea derivatives, thiourea derivatives, cyclic urea and/or thiourea derivatives, poly(ester imide)s, polyesters, esters, guanidine derivatives, cyclic guanidines, imidazoyl compounds, imidazolinyl compounds, imidazolidinyl compounds, cyclic amines, cyclic esters, aromatic rings, bicyclic systems, hydantoins and/or thiohydantoins as well as various other structures. Many types of compounds for the synthesis of peptidomimetic substances are available from a number of commercial sources (e.g. Peptide and Peptidomimetic Synthesis, Reagents for Drug Discovery, Fluka ChemieGmbH, Buchs, Switzerland, 2000 and Novabio-chem 2000 Catalog, Calbiochem-Novabiochem AG, Läufelfingen, Switzerland, 2000). The resemblance between the peptidomimetic compounds and the original peptides is based on structural and/or functional similarities. Thus, the peptidomimetic compounds mimic the properties of the original peptides and, for the purpose of the present application, their binding ability is similar to the peptides that they resemble. Peptidomimetic compounds can be made up, for example, of unnatural amino acids (such as D-amino acids or amino acids comprising unnatural side chains, or of beta-amino acids etc.), which do not appear in the original peptides, or they can be considered to consist of or can be made from other compounds or structural units. Examples of synthetic peptidomimetic compounds comprise N-alkylamino cyclic urea, thiourea, polyesters, poly(ester imide)s, bicyclic guanidines, hydantoins, thiohydantoins, and imidazol-pyridino-inoles (Houghten et al. 1999, ibid. and Nargund et al., 1998, ibid.). Such peptidomimetic compounds can be characterized as being “structural or functional analogues” of the peptides of this invention.

For the purpose of the present invention, the term “targeting unit” stands for a compound, a peptide or a structural or functional analogue thereof, capable of selectively targeting and selectively binding to tumor tissue, tumors, and, preferably, also to tumor stroma, tumor parenchyma and/or extracellular matrix (ECM) of tumors. More specifically, the targeting units may bind to a cell surface, to a specific molecule, molecular complex or structure on a cell surface or within the cells, extracellularly in the vicinity of cells or they may associate with the extracellular matrix present between the cells. The targeting units may also bind to the endothelial cells or the extracellular matrix of tumor vasculature. The targeting units may bind also to the tumor mass, tumor cells and extracellular matrix of metastases.

Generally, the terms “targeting” or “binding” stand for adhesion, at-attachment, affinity or binding of the targeting units of this invention to tumors, tumor cells and/or tumor tissue to the extent that the binding can be objectively measured and determined e.g., by peptide competition experiments in vivo or ex vivo, on tumor biopsies in vitro or by immunological stainings in situ, or by other methods known by those skilled in the art. Tumor targeting means that the targeting units specifically bind to tumors when administered to a human or animal body. Another term used in the art for this specific association is “homing”. Targeting units and targeting agents according to the present invention are considered to be “bound” to the tumor target in vitro, when the binding is strong enough to withstand normal sample treatment, such as washes and rinses with physiological saline or other physiologically acceptable salt or buffer solutions at physiological pH, or when bound to a tumor target in vivo long enough for the effector unit to exhibit its function on the target.

The binding of the present targeting agents or targeting units to tumors is “selective” meaning that they do not bind to normal cells and organs, or bind to such to a significantly lower degree as compared to tumors. The binding of the present targeting agents to non-cancer cells tested is less than 45% of binding to the cancer cell lines.

Pharmaceutically or physiologically or diagnostically acceptable salts and derivatives of the targeting units and agents of the present invention include e.g. salts, esters, amides, hydrazides, N-substituted amides, N-substituted hydrazides, hydroxamic acid derivatives, decarboxylated and N-substituted derivatives thereof. Other suitable pharmaceutically acceptable derivatives are readily acknowledged by those skilled in the art.

The present invention is based on the finding that a group of linear or cyclic peptides having specific amino acid sequences or motifs are capable of selectively targeting tumors, especially colon primary tumors and metastases, in vivo and tumor cells in vitro. Thus, the peptides of this invention, when administered to a human or animal subject, are capable of selectively binding to tumors but do not bind to normal tissue in the body.

The tumor targeting units according to the present invention were identified by bio-panning of phage display libraries. Phage display is a method whereby libraries of random peptides are expressed on the surface of a bacteriophage as part of the phage capsid protein pill by insertion of its encoding DNA sequence into gene III of the phage genome. The pill libraries display 3-5 copies of each individual peptide per phage particle (Smith and Scott, 1993, Methods Enzymol., 217: 228-257).

Phage display peptide libraries were screened by biopanning to select peptides that are specific to colon cancer. The principle of bio-panning comprises 1) exposing homogenized tissue samples to a phage library, 2) washing off unbound phages, and 3) rescuing the phages bound to the target tissue. Repeating steps 1-3 results in a selection of highly enriched peptides having a high binding affinity towards the target tissue compared to other peptides of the original phage library. In the present invention a phage display peptide library was panned against tissue samples taken from primary tumors of colon cancer patients, as described in more detail in the Examples section.

Targeting Motifs According to the Present Invention

It has now surprisingly been found that a seven-amino-acid motif

| | |
|---|---|
| Y-G-F-X-W-G-Z, | (SEQ ID NO: 26) |

wherein Y is tyrosine, or a structural or functional analogue thereof; G is glycine, or a structural or functional analogue thereof; F is phenylalanine, or a structural or functional analogue thereof; X is alanine, valine, leucine or isoleucine, or a structural or functional analogue thereof; W is tryptophan, or a structural or functional analogue thereof; Z is glutamine or glutamic acid, or a structural or functional analogue thereof; targets and exhibits selective binding to tumors and tumor cells and, especially, to colon/colorectal primary tumors and metastases.

According to the present invention, Y is tyrosine, or a structural or functional analogue thereof characterized either by its ability to structurally mimic tyrosine, for example by virtue of comprising a ring structure of a similar or related type, as compared to the ring structure of tyrosine; or by virtue of comprising another structure that sterically or electrically can be considered as an equivalent of the ring structure of tyrosine. Typically, structural and functional analogues of tyrosine may also be characterized by their ability to mimic the acid-base or electric or bond-conjugation or hydrogen-bond-formation or aromatic or other functional or related properties of tyrosine, e.g. by comprising one or more aromatic rings, one or more hydroxyl groups, often preferably phenolic hydroxyl groups, etc., as is understood by those skilled in the art.

Many types of structural and functional analogues of tyrosine are commercially available, and many more are described in the chemical literature known by those skilled in the art, and further ones can be synthesized by those skilled in the art.

Structural and functional analogues of tyrosine may be selected, for example, from any optical and geometrical isomers of the following non-limiting compounds and their like: 2-fluorotyrosine, 3-fluorotyrosine, 2,3-difluorotyrosine, 2,5-difluorotyrosine, 2,6-difluorotyrosine, 2-chlorotyrosine, 3-chlorotyrosine, 2,3-dichlorotyrosine, 2,5-dichlorotyrosine, 2,6-dichlorotyrosine, 2-bromotyrosine, 3-bromotyrosine, 2,3-dibromotyrosine, 2,5-dibromotyrosine, 2,6-dibromotyrosine, 2-iodotyrosine, 3-iodotyrosine, 2,3-diiodotyrosine, 2,5-diiodotyrosine, 2,6-diiodotyrosine, 2,3,5-trifluorotyrosine, 2,3,6-trifluorotyrosine, 2,3,5,6-tetrafluorotyrosine, 2,3,5-trichlorotyrosine, 2,3,6-trichlorotyrosine, 2,3,5,6-tetrachlorotyrosine, 2,3,5-tribromotyrosine, 2,3,6-tribromotyrosine, 2,3,5,6-tetrabromotyrosine, 2,3,5-triiodotyrosine, 2,3,6-triiodotyrosine, 2,3,5,6-tetraiodotyrosine, other di-, tri- and tetrahalogenated tyrosines, 2-methyltyrosine, 3-methyltyrosine, 2,3-dimethyltyrosine, 2,5-dimethyltyrosine, 2,6-dimethyltyrosine, 2-ethyltyrosine, 3-ethyltyrosine, 2,3-diethyltyrosine, 2,5-diethyltyrosine, 2,6-diethyltyrosine, other mono-, di-, tri- and tetraalkylated tyrosines, phosphotyrosine, tyrosine esterified at the phenolic hydroxyl with acetic or fluorocaetic or difluoroacetic or trifluoroacetic or formic or propionic acid or other carboxylic acid, tyrosine etherified at the phenolic hydroxyl with methanol or ethanol or other alcohol, alpha-methyltyrosine, alpha-ethyltyrosine, alpha-propyltyrosine, other alpha-alkylated tyrosines, (2-hydroxyphenyl)-alanine, (3-hydroxyphenyl)-alanine, (2,3-dihydroxyphenyl)-alanine, (2,4-dihydroxyphenyl)-alanine, (2,5-dihydroxyphenyl)-alanine, (2,6-dihydroxyphenyl)-alanine, (3,4-dihydroxyphenyl)-alanine, (3,5-dihydroxyphenyl)-alanine, trihydroxyphenyl-alanines, tetrahydroxyphenyl-alanines, pentahydroxyphenyl-alanine, (ring-hydroxyl)-esterified forms of said hydroxylated phenyalanines (both those esterified at all hydroxyl functions and those not esterified at all hydroxyl functions), 4-(4-hydroxyphenyl)-2-aminobutanoic acid, other tyrosine analogues comprising a longer aliphatic part between the carboxyl function and the aromatic ring than in tyrosine, 3-[3-hydroxy-(1-naphtyl)]-2-aminopropionic acid and its analogues carrying the phenolic hydroxyl at another position in the naphtyl ring system or carrying more than one hydroxyl functions in the naphtyl ring system, 3-(4-hydroxycyclohexyl)-2-aminopropionic acid and its analogues carrying the alcoholic hydroxyl at another position in the ring system or carrying more than one hydroxyl functions in the ring system.

According to the present invention, G is glycine, or a structural of functional analogue thereof characterized by its ability to structurally mimic glycine for example by virtue of comprising a unit of minimal size, as compared to almost any other amino acid, by virtue of its lack of any highly bulky groups and structural fragments and parts and side chains that would cause marked steric hindrance and crowding, by virtue of its lack of aromatic or other ring structures, or by virtue of being otherwise sterically or electrically equivalent to the structure of glycine, e.g. by virtue of having only a very small side chain, or by virtue of having no marked or pronounced lipohilicity or hydrophobicity, or by virtue of having no pronounced unsaturated character or aromatic character, or by having no special structural rigidity such as that of unsaturated structures.

Suitable structures may, for example, preferably comprise a small side chain such as a methyl or an ethyl group or their like or a halogenated methyl or ethyl group etc., or may be totally devoid of any side chain, which may also be preferable. Even a small monocyclic structure may be included, such as cyclopropyl group, but at least very large rings should be completely avoided. Such a monocyclic structure may or may not comprise also one or more heteroatom(s) or substituents etc. Suitable structures may, for example, also be beta-amino acids, gamma-amino acids etc. instead of being natural alpha-amino acids, and also in that case may for example comprise a small side chain such as a methyl or an ethyl group or their like or a halogenated methyl or ethyl group etc. or may be totally devoid of any side chain. Of course, also in the case of structural and functional analogues of glycine, the analogues need not be amino acids at all but may be for example amino alcohols, amino sugars, amino ketones etc. or may be devoid of any amino group, and so on, as is understood by those skilled in the art.

Many types of structural and functional analogues of glycine are commercially available, and many more are described in the chemical literature known by those skilled in the art, and further ones can be synthesized by those skilled in the art.

Structural and functional analogues of glycine may be selected, for example, from any optical and geometrical isomers of the following non-limiting compounds and their like: 2-aminopropanoic acid, 3-aminopropanoic acid, 2-aminobutanoic acid, 3-aminobutanoic acid, 4-aminobutanoic acid, 2,3-diaminopropanoic acid, 2,3-diaminobutanoic acid, 3,4-diaminobutanoic acid, 2,4-diaminobutanoic acid, 2-amino-2-methylpropanoic acid, 3-amino-2-methylpropanoic acid, 2-amino-2-methylbutanoic acid, 3-amino-2-methylbutanoic acid, 4-amino-2-methylbutanoic acid, 2,3-diamino-2-methylpropanoic acid, 2,3-d amino-2-methylbutanoic acid, 3,4-diamino-2-methylbutanoic acid, 2,4-diamino-2-methylbutanoic acid, 2-amino-3-methylbutanoic acid, 3-amino-3-methylbutanoic acid, 4-amino-3-methylbutanoic acid, 2,3-diamino-3-methylpropanoic acid, 2,3-diamino-3-methylbutanoic acid, 3,4-diamino-3-methylbutanoic acid, 2,4-diamino-3-methylbutanoic acid, 2-amino-2-ethylbutanoic acid, 3-amino-2-ethylbutanoic acid, 4-amino-2-ethylbutanoic acid, 2,3-diamino-2-ethylpropanoic acid, 2,3-diamino-2-ethylbutanoic acid, 3,4-diamino-2-ethylbutanoic acid, 2,4-diamino-2-ethylbutanoic acid, 2-amino-3-ethylbutanoic acid, 3-amino-3-ethylbutanoic acid, 4-amino-3-ethylbutanoic acid, 2,3-diamino-3-ethylpropanoic acid, 2,3-diamino-3-ethylbutanoic acid, 3,4-diamino-3-ethylbutanoic acid, 2,4-diamino-3-ethylbutanoic acid, 2-amino-2-cyclopropylethanoic acid, 2-amino-2-cyclopropylpropanoic acid, 3-amino-2-cyclopropylpropanoic acid, 2-amino-2-cyclopropylbutanoic acid, 3-amino-2-cyclopropylbutanoic acid, 4-amino-2-cyclopropylbutanoic acid, 2,3-diamino-2-cyclopropylpropanoic acid, 2,3-diamino-2-cyclopropylbutanoic acid, 3,4-diamino-2-cyclopropylbutanoic acid, 2,4-diamino-2-cyclopropylbutanoic acid, 2-amino-3-cyclopropylbutanoic acid, 3-amino-3-cyclopropylbutanoic acid, 4-amino-3-cyclopropylbutanoic acid, 2,3-diamino-3-cyclopropylpropanoic acid, 2,3-diamino-3-cyclopropylbutanoic acid, 3,4-d amino-3-cyclopropylbutanoic acid, 2,4-diamino-3-cyclopropylbutanoic acid, 2-amino-2-(fluorocyclopropyl)-ethanoic acid, 2-amino-2-(fluorocyclopropyl)-propanoic acid, 3-amino-2-(fluorocyclopropyl)-propanoic acid, 2-amino-2-(fluorocyclopropyl)-butanoic acid, 3-amino-2-(fluorocyclopropyl )-butanoic acid, 4-amino-2-(fluorocyclopropyl)-butanoic acid, 2,3-diamino-2-(fluorocyclopropyl)-propanoic acid, 2,3-diamino-2-(fluorocyclopropyl)-butanoic acid, 3,4-diamino-2-(fluorocyclopropyl)-butanoic acid, 2,4-diamino-2-(fluorocyclopropyl)-butanoic acid, 2-amino-3-(fluorocyclopropyl)-butanoic acid, 3-amino-3-(fluorocyclopropyl)-butanoic acid, 4-amino-3-(fluorocyclopropyl)-butanoic acid, 2,3-diamino-3-(fluorocyclopropyl )-propanoic acid, 2,3-diamino-3-(fluorocyclopropyl)-butanoic acid, 3,4-diamino-3-(fluorocyclopropyl)-butanoic acid, and 2,4-diamino-3-(fluorocyclopropyl)-butanoic acid.

According to the present invention, F is phenylalanine, or a structural or functional analogue thereof, characterized by its ability to structurally mimic phenylalanine for example by virtue of comprising a ring structure of a similar or related type, as compared to the ring structure of phenylalanine, or by virtue of comprising another structure that sterically or electrically can be considered as an equivalent of the ring structure, e.g. by virtue of comprising at least one unsaturated bond that render structural rigidity as compared to saturated structures. Said ring structures may preferably comprise a monocyclic structure that may or may not comprise also one or more heteroatom(s), or may be for example bicyclic or tricyclic. Also ring structures comprising at least one substituent may be employed, etc., as is understood by those skilled in the art. Typically, structural and functional analogues of phenylalanine may also be characterized by their ability to mimic the electric or bond-conjugation or aromatic or steric or other functional or related properties of phenylalanine, e.g. by comprising at least one aromatic ring, etc., as is understood by those skilled in the art.

Many types of structural and functional analogues of phenylalanine are commercially available, and many more are described in the chemical literature known by those skilled in the art, and further ones can be synthesized by those skilled in the art.

Structural and functional analogues of phenylalanine may be selected, for example, from any optical and geometrical isomers of the following non-limiting compounds and their like: phenylalanine, 2-amino-4phenylbutanoic acid, 3-amino-4-phenylbutanoic acid, 2-amino-3-phenyl-butanoic acid, 2-amino-5-phenylpentanoic acid, 3-amino-5-phenylpentanoic acid, 4-amino-5-phenylpentanoic acid, 2-amino-4-phenylpentanoic acid, 3-amino-4-phenylpentanoic acid, 2-amino-3-phenylpentanoic acid, 2-amino-6-phenylhexanoic acid, 3-amino-6-phenylhexanoic acid, 4-amino-6-phenylhexanoic acid, 2-amino-5-phenylhexanoic acid, 2-amino-4-phenylhexanoic acid, 2-amino-3-phenylhexanoic acid, 2-amino-4-phenylhexanoic acid, 3-amino-4-phenylhexanoic acid, 2-amino-3-phenylhexanoic acid, 2-amino-4-(1-naphtyl)butanoic acid, 3-amino-4-(1-naphtyl)butanoic acid, 2-amino-3-(1-naphtyl)-butanoic acid, 2-amino-5-(1-naphtyl)pentanoic acid, 3-amino-5-(1-naphtyl)-pentanoic acid, 4-amino-5-(1-naphtyl)pentanoic acid, 2-amino-4-(1-naphtyl)-pentanoic acid, 3-amino-4-(1-naphtyl)pentanoic acid, 2-amino-3-(1-naphtyl)-pentanoic acid, 2-amino-6-(1-naphtyl)hexanoic acid, 3-amino-6-(1-naphtyl)-hexanoic acid, 4-amino-6-(1-naphtyl)hexanoic acid, 2-amino-5-(1-naphtyl)-hexanoic acid, 2-amino-4-(1-naphtyl)hexanoic acid, 2-amino-3-(1-naphtyl)-hexanoic acid, 2-amino-4-(1-naphtyl)hexanoic acid, 3-amino-4-(1-naphtyl)-hexanoic acid, 2-amino-3-(1-naphtyl)hexanoic acid, 2-amino-4-(2-naphtyl)-butanoic acid, 3-amino-4-(2-naphtyl)butanoic acid, 2-amino-3-(2-naphtyl)-butanoic acid, 2-amino-5-(2-naphtyl)pentanoic acid, 3-amino-5-(2-naphtyl)-pentanoic acid, 4-amino-5-(2-naphtyl)pentanoic acid, 2-amino-4-(2-naphtyl)-pentanoic acid, 3-amino-4-(2-naphtyl)pentanoic acid, 2-amino-3-(2-naphtyl)-pentanoic acid, 2-amino-6-(2-naphtyl)hexanoic acid, 3-amino-6-(2-naphtyl)-hexanoic acid, 4-amino-6-(2-naphtyl)hexanoic acid, 2-amino-5-(2-naphtyl)-hexanoic acid, 2-amino-4-(2-naphtyl)hexanoic acid, 2-amino-3-(2-naphtyl)-hexanoic acid, 2-amino-4-(2-naphtyl)hexanoic acid, 3-amino-4-(2-naphtyl)-hexanoic acid, 2-amino-3-(2-naphtyl)hexanoic acid, 2-amino-4-(2-methyl-phenyl)butanoic acid, 3-amino-4-(2-methylphenyl)butanoic acid, 2-amino-3-(2-methylphenyl)butanoic acid, 2-amino-5-(2-methylphenyl)pentanoic acid, 3-amino-5-(2-methylphenyl)pentanoic acid, 4-amino-5-(2-methylphenyl)pentanoic acid, 2-amino-4-(2-methylphenyl)pentanoic acid, 3-amino-4-(2-methylphenyl)pentanoic acid, 2-amino-3-(2-methylphenyl)pentanoic acid, 2-amino-6-(2-methylphenyl)hexanoic acid, 3-amino-6-(2-methylphenyl)hexanoic acid, 4-amino-6-(2-methylphenyl)hexanoic acid, 2-amino-5-(2-methylphenyl)hexanoic acid, 2-amino-4-(2-methylphenyl)hexanoic acid, 2-amino-3-(2-methylphenyl)-hexanoic acid, 2-amino-4-(2-methylphenyl)hexanoic acid, 3-amino-4-(2-methylphenyl)hexanoic acid, 2-amino-3-(2-methylphenyl)hexanoic acid, 2-amino-4-(3-methylphenyl)butanoic acid, 3-amino-4-(3-methylphenyl)butanoic acid, 2-amino-3-(3-methylphenyl)butanoic acid, 2-amino-5-(3-methylphenyl)pentanoic acid, 3-amino-5-(3-methylphenyl)pentanoic acid, 4-amino-5-(3-methyl-phenyl)pentanoic acid, 2-amino-4-(3-methylphenyl)pentanoic acid, 3-amino-4-(3-methylphenyl)pentanoic acid, 2-amino-3-(3-methylphenyl)pentanoic acid, 2-amino-6-(3-methylphenyl )hexanoic acid, 3-amino-6-(3-methylphenyl)hexanoic acid, 4-amino-6-(3-methylphenyl)hexanoic acid, 2-amino-5-(3-methylphenyl)-hexanoic acid, 2-amino-4-(3-methylphenyl)hexanoic acid, 2-amino-3-(3-methylphenyl)hexanoic acid, 2-amino-4-(3-methylphenyl)hexanoic acid, 3-amino-4-(3-methylphenyl)hexanoic acid, 2-amino-3-(3-methylphenyl)hexanoic acid, 2-amino-4-phenylbutanoic acid, 3-amino-4-phenylbutanoic acid, 2-amino-3-phenylbutanoic acid, 2-amino-5-phenylpentanoic acid, 3-amino-5-phenyl-pentanoic acid, 4-amino-5-phenylpentanoic acid, 2-amino-4-phenylpentanoic acid, 3-amino-4-phenylpentanoic acid, 2-amino-3-phenylpentanoic acid, 2-amino-6-phenylhexanoic acid, 3-amino-6-phenylhexanoic acid, 4-amino-6-phenyl-hexanoic acid, 2-amino-5-phenylhexanoic acid, 2-amino-4-phenylhexanoic acid, 2-amino-3-phenylhexanoic acid, 2-amino-4-phenylhexanoic acid, 3-amino-4-phenylhexanoic acid, and 2-amino-3-phenylhexanoic acid.

In one preferred embodiment of the invention, X is alanine, or a structural or functional analogue thereof. Such an analogue may preferably have no side chain or may comprise in its side chain(s) maximally four, more preferably maximally three, still more preferably maximally two, non-hydrogen atoms. Structural or functional analogues of alanine include for example any optical isomers of compounds such as: 3-chloroalanine, 3-fluoroalanine, 2-aminobutanoic acid, 4-fluoro-2-aminobutanoic acid, 4-chloro-2-aminobutanoic acid, 3-cyanoalanine, 3-cyclopropylalanine, 2-amino-3-butenoic acid and 2-amino-3-butynoic acid.

In another preferred embodiment according to the present invention, X is valine or leucine or isoleucine, or a structural or functional analogue thereof. Such an analogue may for example be characterized by its ability to structurally mimic valine, leucine or isoleucine, for example by virtue of comprising at least one aliphatic, cycloaliphatic/alicyclic, or related side-chain or, generally, at least one side-chain comprising at least one hydrophobic structure or group or lipophilic structure or group, or generally by virtue of comprising at least one small side-chain that do not cause massive sterical hindrance, etc., as is understood by those skilled in the art. Typically, structural and functional analogues of valine, leucine and isoleucine may preferably be characterized by more than one of said features or properties.

Many types of structural and functional analogues of valine and leucine and isoleucine are commercially available, and many more are described in the chemical literature known by those skilled in the art, and further ones can be synthesized by those skilled in the art.

Structural and functional analogues of valine and leucine and isoleucine may be selected, for example, from any optical and geometrical isomers of the following compounds and their like: alanine, valine, leucine, isoleucine, norleucine, norvaline, allo-isoleucine, 2-aminobutanoic acid, 2-amino-2-methylpropionic acid, 2-amino-4,4-dimethylpentanoic acid, 4,5-dehydroleucine, 2-amino-6-isopropylamino-hexanoic acid, 4-amino-6-methylheptanoic acid, 3-amino-6-methylheptanoic acid, 2-amino-6-methylheptanoic acid, tert-leucine, 4-amino-5-cyclohexyl-3-hydroxypentanoic acid, 4-amino-5-cyclohexylpentanoic acid, 2-amino-2-cyclohexylacetic acid, 2-amino-3-cyclohexylpropionic acid, 2-amino-4-cyclohexylbutanoic acid, 2-amino-3-cyclopentylpropionic acid, 2-amino-4-cyclopentylbutanoic acid, 2-amino-3-cyclobutylpropionic acid, 2-amino-4-cyclobutylbutanoic acid, 2-amino-3-cyclopropylpropionic acid, 2-amino-4-cyclopropylbutanoic acid, 2-amino-3-(1-cyclopentenyl)-propionic acid, 2-amino-4-(1-cyclopentenyl)-butanoic acid, 2-amino-3-ethylsulfanylpropionic acid, 2-amino-3-methylsulfanylpropionic acid, 3-fluoroalanine, 3-chloroalanine, 3,3-dicyclohexylalanine, 2-amino-3-propenoic acid, 2-amino-4,4-dimethylpentanoic acid or statine,

or from the group consisting of any N-methyl analogues of any one of the aforementioned, any N-ethyl analogues of any of the aforementioned, any other N-alkyl analogues of any of the aforementioned, any alpha-methyl analogues (2-methyl-analogues) of any of the aforementioned, any alpha-ethyl analogues (2-ethyl analogues) of any of the aforementioned, and any other alpha-alkyl analogues (2-alkyl analogues) of any of the aforementioned;

or from the group consisting of any analogues of the aforementioned comprising in a side chain a branched, non-branched and/or alicyclic structure with at least two similar or different atoms selected from the group of carbon atoms, silicon atoms, halogen atoms bonded to at least one carbon, ether-oxygens and thioether-sulphurs;

or, more generally, from the group consisting of branched, non-branched and/or cyclic non-aromatic, lipophilic and/or hydrophobic amino acids and amino acid analogues and derivatives and structural or functional analogues thereof, and of amino acids and carboxylic acids and amino acid analogues and derivatives and carboxylic acid analogues and derivatives that have at least one lipophilic carborane-type or other lipophilic boron-containing side chain or its equivalent or other lipophilic cage-type structure.

According to the present invention, W is tryptophan, or a structural or functional analogue thereof, characterized by its ability to structurally mimic tryptophan, for example by virtue of comprising a ring structure of a similar or related type, as compared to the ring structure of tryptophan, or by virtue of comprising another structure that sterically or electrically can be considered as an equivalent of the ring structure, e.g. by virtue of at least one unsaturated bond that render structural rigidity as compared to unsaturated structures. Such ring structures may preferably comprise a bicyclic structure that may or may not comprise nitrogen, or may be for example monocyclic or tricyclic. Also ring structures not comprising nitrogen, or comprising more than one nitrogen atoms, may be employed, etc., as is understood by those skilled in the art. Typically, structural and functional analogues of tryptophan may often also be characterized by their ability to mimic the acid-base or electric or bond-conjugation or aromatic or other functional or related properties of tryptophan, e.g. by comprising one or more aromatic rings, one or more nitrogen atoms, etc., as is understood by those skilled in the art.

Many types of structural and functional analogues of tryptophan are commercially available, and many more are described in the chemical literature known by those skilled in the art, and further ones can be synthesized by those skilled in the art.

Structural and functional analogues of tryptophan may be selected, for example, from any optical and geometrical isomers of the following non-limiting compounds and their like: 2-amino-3-(1-indolyl)-propionic acid, 2-amino-3-(2-indolyl)-propionic acid, 2-amino-3-(4-indolyl)-propionic acid, 2-amino-3-(5-indolyl)-propionic acid, 2-amino-3-(6-indolyl)-propionic acid, 2-amino-3-(7-indolyl)-propionic acid, 2-amino-4-(1-indolyl)-butyric acid, 2-amino-4-(2-indolyl)-butyric acid, 2-amino-4-(3-indolyl)-butyric acid, 2-amino-4-(4-indolyl)-butyric acid, 2-amino-4-(5-indolyl)-butyric acid, 2-amino-4-(6-indolyl)-butyric acid, 2-amino-4-(7-indolyl)-butyric acid, 2-amino-3-(1-benzimidazolyl)-propionic acid, 2-amino-3-(2-benzimidazolyl)-propionic acid, 2-amino-3-(4-benzimidazolyl)-propionic acid, 2-amino-3-(5-benzimidazolyl)-propionic acid, 2-amino-3-(6-benzimidazolyl)-propionic acid, 2-amino-3-(7-benzimidazolyl)-propionic acid, 2-amino-4-(1-benzimidazolyl)-butyric acid, 2-amino-4-(2-benzimidazolyl)-butyric acid, 2-amino-4-(4-benzimidazolyl)-butyric acid, 2-amino-4-(5-benzimidazolyl)-butyric acid, 2-amino-4-(6-benzimidazolyl)-butyric acid, 2-amino-4-(7-benzimidazolyl)-butyric acid, 2-amino-3-(1-indenyl)-propionic acid, 2-amino-3-(2-indenyl)-propionic acid, 2-amino-3-(3-indenyl)-propionic acid, 2-amino-3-(4-indenyl)-propionic acid, 2-amino-3-(5-indenyl)-propionic acid, 2-amino-3-(6-indenyl)-propionic acid, 2-amino-3-(7-indenyl)-propionic acid, 2-amino-3-(8-indenyl)-propionic acid, 2-amino-4-(1-indenyl)-butyric acid, 2-amino4-(2-indenyl)-butyric acid, 2-amino-4-(3-indenyl)-butyric acid, 2-amino-4-(4-indenyl)-butyric acid, 2-amino-4-(5-indenyl)-butyric acid, 2-amino-4-(6-indenyl)-butyric acid, 2-amino-4-(7-indenyl)-butyric acid, 2-amino-4-(8-indenyl)-butyric acid, 2-amino-3-(2-purinyl)-propionic acid, 2-amino-3-(6-purinyl)-propionic acid, 2-amino-3-(8-purinyl)-propionic acid, 2-amino-3-(9-purinyl)-propionic acid, 2-amino-4-(2-purinyl)-butyric acid, 2-amino-4-(6-purinyl)-butyric acid, 2-amino-4-(8-purinyl)-butyric acid, 2-amino-4-(9-purinyl)-butyric acid, 2-amino-3-(2-benzothienyl)-propionic acid, 2-amino-3-(3-benzothienyl)-propionic acid, 2-amino-3-(4-benzothienyl)-propionic acid, 2-amino-3-(5-benzothienyl)-propionic acid, 2-amino-3-(6-benzothienyl )-propionic acid, 2-amino-3-(7-benzothienyl)-propionic acid, 2-amino-4-(2-benzothienyl)-butyric acid, 2-amino-4-(3-benzothienyl)-butyric acid, 2-amino-4-(4-benzothienyl)-butyric acid, 2-amino-4-(5-benzothienyl)-butyric acid, 2-amino-4-(6-benzothienyl)-butyric acid, 2-amino-4-(7-benzothienyl)-butyric acid, 2-amino-3-(1-naphtyl)-propionic acid, 2-amino-3-(2-naphtyl)-propionic acid, 2-amino-4-(1-naphtyl)-butyric acid, 2-amino-4-(2-naphtyl)-butyric acid, 2-amino-3-(2-pyridyl)-propionic acid, 2-amino-3-(3-pyridyl)-propionic acid, 2-amino-3-(4-pyridyl)-propionic acid, 2-amino-4-(2-pyridyl)-butyric acid, 2-amino-4-(3-pyridyl)-butyric acid, 2-amino-4-(4-pyridyl)-butyric acid, 2-amino-3-(1-pyrrolyl)-propionic acid, 2-amino-3-(2-pyrrolyl)-propionic acid, 2-amino-3-(3-pyrrolyl)-propionic acid, 2-amino-3-(4-pyrrolyl)-propionic acid, 2-amino-4-(1-pyrrolyl)-butyric acid, 2-amino-4-(2-pyrrolyl)-butyric acid, 2-amino-4-(3-pyrrolyl)-butyric acid, 2-amino-4-(4-pyrrolyl)-butyric acid, 2-amino-3-(2-pyridyl)-propionic acid, 2-amino-3-(3-pyridyl)-propionic acid, 2-amino-3-(4-pyridyl)-propionic acid, 2-amino-4-(2-pyridyl)-butyric acid, 2-amino-4-(3-pyridyl)-butyric acid, 2-amino-4-(4-pyridyl)-butyric acid, 2-amino-3-(3-pyridazinyl)-propionic acid, 2-amino-3-(4-pyridazinyl)-propionic acid, 2-amino-4-(3-pyridazinyl)-butyric acid, 2-amino-4-(4-pyridazinyl)-butyric acid, 2-amino-3-(2-pyrimidinyl)-propionic acid, 2-amino-3-(4-pyrimidinyl)-propionic acid, 2-amino-3-(5-pyrimidinyl)-propionic acid, 2-amino-3-(6-pyrimidinyl)-propionic acid, 2-amino-4-(2-pyrimidinyl)-butyric acid, 2-amino-4-(4-pyrimidinyl)-butyric acid, 2-amino-4-(5-pyrimidinyl)-butyric acid, 2-amino-4-(6-pyrimidinyl)-butyric acid, 2-amino-3-(I-pyrrolyl)-propionic acid, 2-amino-3-(2-pyrrolyl)-propionic acid, 2-amino-3-(3-pyrrolyl)-propionic acid, 2-amino-4-(1-pyrrolyl)-butyric acid, 2-amino-4-(2-pyrrolyl)-butyric acid, 2-amino-4-(3-pyrrolyl)-butyric acid, 2-amino-3-(1-pyrrolinyl)-propionic acid, 2-amino-3-(2-pyrrolinyl)-propionic acid, 2-amino-3-(3-pyrrolinyl)-propionic acid, 2-amino-4-(1-pyrrolinyl)-butyric acid, 2-amino-4-(2-pyrrolinyl)-butyric acid, 2-amino-4-(3-pyrrolinyl)-butyric acid, 2-amino-3-(1-pyrrolidinyl)-propionic acid, 2-amino-3-(2-pyrrolidinyl)-propionic acid, 2-amino-3-(3-pyrrolidinyl)-propionic acid, 2-amino-4-(1-pyrrolidinyl)-butyric acid, 2-amino-4-(2-pyrrolidinyl)-butyric acid, 2-amino-4-(3-pyrrolidinyl)-butyric acid, 2-amino-3-(1-pyrazolyl)-propionic acid, 2-amino-3-(3-pyrazolyl)-propionic acid, 2-amino-3-(4-pyrazolyl)-propionic acid, 2-amino-3-(5-pyrazolyl)-propionic acid, 2-amino-4-(1-pyrazolyl )-butyric acid, 2-amino-4-(3-pyrazolyl)-butyric acid, 2-amino-4-(4-pyrazolyl)-butyric acid, 2-amino-4-(5-pyrazolyl)-butyric acid, 2-amino-3-(1-pyrazolinyl)-propionic acid, 2-amino-3-(3-pyrazolinyl)-propionic acid, 2-amino-3-(4-pyrazolinyl)-propionic acid, 2-amino-3-(5-pyrazolinyl)-propionic acid, 2-amino-4-(1-pyrazolinyl)-butyric acid, 2-amino-4-(3-pyrazolinyl)-butyric acid, 2-amino-4-(4-pyrazolinyl)-butyric acid, 2-amino-4-(5-pyrazolinyl)-butyric acid, 2-amino-3-(1-pyrazolidinyl)-propionic acid, 2-amino-3-(2-pyrazolidinyl)-propionic acid, 2-amino-3-(3-pyrazolidinyl)-propionic acid, 2-amino-3-(4-pyrazolidinyl)-propionic acid, 2-amino-3-(5-pyrazolidinyl)-propionic acid, 2-amino-4-(1-pyrazolidinyl)-butyric acid, 2-amino-4-(2-pyrazolidinyl)-butyric acid, 2-amino-4-(3-pyrazolidinyl)-butyric acid, 2-amino-4-(4-pyrazolidinyl)-butyric acid, 2-amino-4-(5-pyrazolidinyl)-butyric acid, 2-amino-3-(1-imidazolyl)-propionic acid, 2-amino-3-(2-imidazolyl)-propionic acid, 2-amino-3-(4-imidazolyl)-propionic acid, 2-amino-3-(5-imidazolyl)-propionic acid, 2-amino-4-(1-imidazolyl)-butyric acid, 2-amino-4-(2-imidazolyl)-butyric acid, 2-amino-4-(4-imidazolyl)-butyric acid, 2-amino-4-(5-imidazolyl)-butyric acid;

or from the group consisting of any substituted and unsubstituted acetic, valeric and other branched and non-branched carboxylic acid analogues of any of said propionic and/or butyric acids;

or from the group consisting of any amino acids and carboxylic acids comprising in at least one side chain or as at least one side chain: at least one indene, naphthalene, benzofuran, indole, benzo[b]thiophene, benzimidazole, benzothiazole, purine, quinoline, isoquinoline, cinnoline, quinoxaline, azulene, fluorene, dibenzofuran, carbazole, anthracene, phenathrene, acridine, 1,10-phenanthroline, phenothiazine, pyrene, furan, pyrrole, 3-pyrroline, pyrrolidine, pyrazole, 2-pyrazoline, pyrazolidine, imidazole, oxazole, thiazole, 1,2,3-oxadiazole, 1,2,3-triazole, 1,2,4-triazole, 1,3,4-thiadiazole, pyridine, pyridazine, pyrimidine, pyradine, piperazine and/or 1,3,5-triazine ring/system/radical/substituent;

or from the group consisting of any carboxylic acids and amino acids that comprise in at least one side chain or as at least one side chain: at least one ring selected from the group of: bicyclic structures comprising one aromatic or heteroaromatic 6-ring and one 5-ring comprising at least one nitrogen atom, bicyclic structures comprising at least one aromatic or heteroaromatic 6-ring, tricyclic structures comprising one aromatic or heteroaromatic 6-ring and one 5-ring comprising at least one nitrogen atom, tricyclic structures comprising at least one aromatic or heteroaromatic 6-ring, bicyclic structures comprising at least one 5-ring comprising at least one nitrogen atom, tricyclic structures comprising at least one 5-ring comprising at least one nitrogen atom, tetracyclic structures comprising at least one aromatic and/or heteroaromatic ring and/or at least one ring comprising at least one nitrogen atom, monocyclic structures comprising at least one nitrogen atom, other aromatic and heteroaromatic structures and structures comprising at least one aromatic or pseudoaromatic ring, other structures comprising at least one 4- or 5- or 6- or 7-ring that comprises at least one nitrogen atom.

According to one preferred embodiment of the present invention, Z is glutamine, or a structural or functional analogue thereof. Typically, structural and functional analogues of glutamine may be characterized by their ability to structurally mimic glutamine for example by virtue of comprising in a side chain or in more than one side chains a carboxylamide (an amide) group or a structure of a similar or related type, as compared to the carboxylamide structure of glutamine, or by virtue of comprising another structure that sterically or electrically can be considered as an equivalent of the amide structure. One possibility is to use an unsubstituted carboxylamide function similar to that in glutamine (or a substituted one, or more than one carboxylamide functions, etc.) but in a side chain (or side chains) that is/are otherwise different form the side chain in glutamine, e.g. by virtue of comprising at least one heteroatom instead of at least one carbon atom, or virtue of being longer or shorter than the side chain of glutamine, or by virtue of comprising at least one double or triple bond between carbon atoms, or by comprising substituents such as a fluorine atom or an aromatic ring or an alkyl group or a hydroxyl group etc., or by virtue of being branched, etc., as is understood by those skilled in the art. Typically, structural and functional analogues of glutamine may also be characterized by their ability to mimic the electric or double-bond or steric or other functional or related properties of glutamine, e.g. by comprising at least one C═O or C═N or other double bond, or by comprising at least one carbon with electrical properties similar to those of the carbon in the carboxamide group, or by containing at least one nitrogen atom, etc., as is understood by those skilled in the art. The carboxylamide function of glutamine may be replaced e.g. by an N-substituted or an N,N-disubstituted carboxylamide functionality or by another acylamide functionality or by a carboxylic acyl hydrazide or other acyl hydrazide functionality or a substituted hydrazide group or by a hydroxamic acid function or by a substituted hydroxamic acid function or by an aldoxime group or a ketoxime group or a substituted aldoxime group or a substituted ketoxime group or an aldehyde-derived hydrazone or a ketone-derived hydrazone group, or by other suitable functionality known by those skilled in the art. Also unsubstituted and substituted amidino and guanidino groups may come into question.

Many types of structural and functional analogues of glutamine are commercially available, and many more are described in the chemical literature known by those skilled in the art, and further ones can be synthesized by those skilled in the art.

Structural and functional analogues of glutamine may be selected, for example, from any optical and geometrical isomers of the following non-limiting compounds and their like: glutamine, asparagine, isoglutamine, isoasparagine, beta-hydroxyglutamine, gamma-hydroxyglutamine, beta-hydroxyasparagine, beta-methyleneglutamine, gamma-methyleneglutamine, beta-methyleneasparagine, beta,gamma-dihydroxyglutamine, beta-methylglutamine, gamma-methylglutamine, beta-methylasparagine, beta,gamma-dimethylglutamine, beta-ethylglutamine, gamma-ethylglutamine, beta-ethylasparagine, beta,gamma-diethylglutamine, beta-propylglutamine, gamma-propylglutamine, beta-propylasparagine, beta,gamma-dipropylglutamine, beta-cyclopropylglutamine, gamma-cyclopropylglutamine, beta-cyclopropylasparagine, beta,gamma-dicyclopropylglutamine, beta-(difluoromethyl)-glutamine, gamma-(difluoromethyl)-glutamine, beta-(difluoromethyl)-asparagine, beta,gamma-bis(difluoromethyl)-glutamine, 2,6-diamino-6-oxo-hexanoic acid, 2,7-diamino-7-oxo-heptanoic acid, 2,6-diamino-3-methyl-6-oxo-hexanoic acid, 2,7-diamino-3-methyl-7-oxo-heptanoic acid, 2,6-diamino-3-ethyl-6-oxo-hexanoic acid, 2,7-diamino-3-ethyl-7-oxo-heptanoic acid, 2,6-diamino-3-propyl-6-oxo-hexanoic acid, 2,7-diamino-3-propyl-7-oxo-heptanoic acid, 2,6-diamino-3-cyclopropyl-6-oxo-hexanoic acid, 2,7-diamino-3-cyclopropyl-7-oxo-heptanoic acid, 2,6-diamino-3-hydroxy-6-oxo-hexanoic acid, 2,7-diamino-3-hydroxy-7-oxo-heptanoic acid, 2,6-diamino-4-methyl-6-oxo-hexanoic acid, 2,7-diamino-4-methyl-7-oxo-heptanoic acid, 2,6-diamino-4-ethyl-6-oxo-hexanoic acid, 2,7-diamino-4-ethyl-7-oxo-heptanoic acid, 2,6-diamino4-propyl-6-oxo-hexanoic acid, 2,7-diamino-4-propyl-7-oxo-heptanoic acid, 2,6-diamino-4-cyclopropyl-6-oxo-hexanoic acid, 2,7-diamino-4-cyclopropyl-7-oxo-heptanoic acid, 2,6-diamino-4-hydroxy-6-oxo-hexanoic acid, 2,7-diamino-4-hydroxy-7-oxo-heptanoic acid, 2,6-diamino-5-methyl-6-oxo-hexanoic acid, 2,7-diamino-5-methyl-7-oxo-heptanoic acid, 2,6-diamino-5-ethyl-6-oxo-hexanoic acid, 2,7-diamino-5-ethyl-7-oxo-heptanoic acid, 2,6-diamino-5-propyl-6-oxo-hexanoic acid, 2,7-diamino-5-propyl-7-oxo-heptanoic acid, 2,6-diamino-5-cyclopropyl-6-oxo-hexanoic acid, 2,7-diamino-5-cyclopropyl-7-oxo-heptanoic acid, 2,6-diamino-5-hydroxy-6-oxo-hexanoic acid, 2,7-diamino-5-hydroxy-7-oxo-heptanoic acid, as well as from the (amide-N)-monomethylated, (amide-N)-monoethylated, (amide-N)-monopropylated, other (amide-N)-monomalkylated and (amide-N)-dialkylated derivatives of any of the aforementioned compounds.

In another preferred embodiment according to the present invention, Z is glutamic acid, or a structural or functional analogue thereof comprising at least one oxygen atom capable of hydrogen bond formation, and preferably comprising at least one carboxyl group, esterified carboxyl group, hydroxamic acid function, esterified hydroxamic acid function, alcoholic or phenolic hydroxyl group, esterified alcoholic or phenolic hydroxyl group, keto group or aldehyde function, and more preferably comprising at least one carboxyl group, esterified carboxyl group, hydroxamic acid function, esterified hydroxamic acid function, alcoholic or phenolic hydroxyl group or esterified alcoholic or phenolic hydroxyl group, still more preferably comprising at least one carboxyl group, esterified carboxyl group, hydroxamic acid function, alcoholic hydroxyl group or esterified alcoholic hydroxyl group, and most preferably comprising at least one carboxyl group or esterified carboxyl group; or comprising one or more other oxo acid functional groups, selected preferably from the group of: —SO₃⁻, —OSO₃⁻, any inorganic phosphate group or its ester.

Many further types of structural and functional analogues of glutamic acid are commercially available, and many more are described in the chemical literature known by those skilled in the art, and further ones can be synthesized by those skilled in the art. Such analogues may be selected, for example, from any optical and geometrical isomers of the following non-limiting compounds and their like: aspartic acid, 2-amino-1,6-heptanedioic acid, 3-amino-1,6-heptanedioic acid, 2-amino-3-methyl-1,6-heptanedioic acid, 3-amino-2-methyl-1,6-heptanedioic acid, 2-amino-4-methyl-1,6-heptanedioic acid, 2-amino-1,7-hexanedioic acid, 3-amino-1,7-hexanedioic acid, 2-amino-3-methyl-1,7-hexanedioic acid, 3-amino-2-methyl-1,7-hexanedioic acid, 2-amino-4-methyl-1,7-hexanedioic acid, 4-amino-1,7-hexanedioic acid, 4-amino-3-methyl-1,7-hexanedioic acid, 2-amino-4-methyl-1,7-hexanedioic acid, 3-amino-4-methyl-1,7-hexanedioic acid, 2-amino-5-methyl-1,7-hexanedioic acid, 3-methyl-aspartic acid, 3-methyl-glutamic acid, 2-amino-4-phenyl-1,6-heptanedioic acid, 3-amino-4-phenyl-1,6-heptanedioic acid, 2-amino-3-methyl-3-phenyl-1,6-heptanedioic acid, 3-amino-2-ethyl-1,6-heptanedioic acid, 2-amino-4-ethyl-1,6-heptanedioic acid, 2-amino-4-phenyl-1,7-hexanedioic acid, 3-amino-5-phenyl-1,7-hexanedioic acid, 2-amino-3-ethyl-1,7-hexanedioic acid, 3-amino-2-ethyl-1,7-hexanedioic acid, 2-amino-4-ethyl-1,7-hexanedioic acid, 4-amino-2-phenyl-1,7-hexanedioic acid, 4-amino-3-ethyl-1,7-hexanedioic acid, 2-amino-4-ethyl-1,7-hexanedioic acid, 3-amino-4-ethyl-1,7-hexanedioic acid, 2-amino-5-ethyl-1,7-hexanedioic acid, or from the group consisting of the omega-methyl esters of said compounds, the omega-ethyl esters of said compounds, the omega-cyclopropyl esters of said compounds and the omega hydroxamic acid analogues of said compounds.

A preferred motif according to the present invention is a motif wherein X is valine and Z is glutamic acid, i.e., Y-G-F—V-W-G-E (SEQ ID NO. 1).

Another preferred motif according to the present invention is a motif wherein X is valine and Z is glutamine, i.e., Y-G-F—V-W-G-Q (SEQ ID NO. 2).

Still another preferred motif according to the present invention is a motif wherein X is leucine and Z is glutamine, i.e., Y-G-F-L-W-G-Q (SEQ ID NO. 3).

Yet another preferred motif according to the present invention is a motif wherein X is leucine and Z is glutamic acid, i.e., Y-G-F-L-W-G-E (SEQ ID NO. 4).

Yet another preferred motif according to the present invention is a motif wherein X is alanine and Z is glutamine, i.e., Y-G-F-A-W-G-Q (SEQ ID NO. 5).

Yet another preferred motif according to the present invention is a motif wherein X is alanine and Z is glutamic acid, i.e., Y-G-F-A-W-G-E (SEQ ID NO. 6).

Yet another preferred motif according to the present invention is a motif wherein X is isoleucine and Z is glutamine, i.e., Y-G-F—I—W-G-Q (SEQ ID NO. 7).

Yet another preferred motif according to the present invention is a motif wherein X is isoleucine and Z is glutamic acid, i.e., Y-G-F—I—W-G-E (SEQ ID NO. 8).

The motif Y-G-F—X—W-G-Z (SEQ ID NO: 26) according to the present invention may form part of a larger structure, such as a peptide or some other structure. The compound or structure in question may also comprise more than one motif Y-G-F—X—W-G-Z (SEQ ID NO: 26), and the orientation of the motifs may vary.

Targeting Units According to the Present Invention

It has also been found that peptides, including structural or functional analogues thereof as defined herein, comprising a tumor targeting motif according to the present invention target to and exhibit selective binding to tumors, especially to colon primary tumor and metastases.

Such peptides are highly advantageous for use as targeting units according to the present invention, e.g., because of their small size and their easy, reliable and cheap synthesis. Due to the small size of the peptides according to the present invention, the purification, analysis and quality control is easy and commercially useful.

The targeting units according to the present invention are preferably linear. Linear peptides according to the present invention are fast, easy and cheap to prepare, as they do not require any further processing (cyclization etc.) after synthesis and complicated orthogonal and other protections and extra functional groups are not needed that would be needed for cyclization. It is furthermore easier to link additional units to linear peptides, for example because, there is no need to “reserve” functional groups for the purpose of cyclization, or to use expensive and complicated orthogonal protections, etc. In some preferred embodiments of the present invention, the efficient degradation of linear peptides in the human body is an advantage compared to the use of more slowly degrading substances, e.g., in diagnostic applications where rapid clearance is desired.

In another embodiment of the present invention cyclic peptides are preferred. Thus the targeting units according to the present invention may also be cyclic. Cyclic peptides are usually more stable in vivo and in many other biological systems than are their non-cyclic counterparts, as is known in the art. More stable peptides according to the present invention are highly preferred for certain purposes, for example in certain therapeutic applications.

Preferred targeting units according to the present invention may have at least a sequence

| | |
|---|---|
| C_{y}-Y-G-F-X-W-G-Z-C_{yy} | (SEQ ID NO: 25) |

wherein, Y-G-F—X—W-G-Z (SEQ ID NO: 26) is a tumor targeting motif as defined above, and C_{y} and C_{yy} are optional entities forming a cyclic structure.

Preferred structures are such where C_{y} and C_{yy} are amino acids or analogues thereof containing a thiol group, such as homocysteine or cysteine or analogues thereof, or another structure comprising a thiol group or an oxidized thiol group. One preferred cyclic structure type is characterized by the presence of a disulphide bond (e.g., between cysteine moieties). Non-limiting examples of cyclic structures are, for example, compounds of the formula:

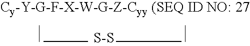

where C_{y}—S—S—C_{yy} indicates a cystine. Because of the easy availability and low price of cysteine, this type of structure is a preferred one.

The —S—S— bridge need not, however, be between cysteine units but may also exist between other amino acids or other moieties containing —SH groups. Such structures may comprise more than one Y-G-F—X—W-G-Z (SEQ ID NO: 26) motif between the cysteine units, and may comprise additional amino acids and structural or functional analogues thereof outside the cyclic structure.

Highly preferred targeting units according to the present invention having a cyclic structure by virtue of a disulphide bridge, are

| | |
|---|---|
| CYGFVWGEC, | (SEQ ID NO. 9) |
| CYGFVWGQC, | (SEQ ID NO. 10) |
| CYGFLWGQC, | (SEQ ID NO. 11) |
| CYGFLWGEC, | (SEQ ID NO. 12) |
| CYGFAWGQC, | (SEQ ID NO. 13) |
| CYGFAWGEC, | (SEQ ID NO. 14) |
| CYGFIWGQC | (SEQ ID NO. 15) |
| and | |
| CYGFIWGEC. | (SEQ ID NO. 16) |

Other preferred possibilities of forming the cyclic structure is the formation of an amide bond to give a lactam, or ester bond to give a lactone, or hydrazone, hydrazine, oxime, thioether or other type of bond to give a cyclic structure.

Lactams, i.e. lactam bridged peptides can be of several subtypes, such as “head to tail”, wherein the ends of the peptide chain are directly linked together (carboxy terminus coupled to amino terminus), “head to side chain” and “side chain to tail”, wherein one end of the peptide chain is linked to side chain of an amino acid residue elsewhere in the peptide (carboxy or amino terminus coupled to one side chain amino or carboxyl group), and “side chain to side chain” (amino group of one side chain coupled to carboxyl group of another side chain).

The terms “C-terminus” and “C-terminal” refer to the carboxylic end of the peptide chain, which may be free, or coupled to another moiety. Moreover, The terms “N-terminus” and “N-terminal” refer to the amino end of the peptide chain, which may be free, or coupled to another moiety.

Preferred structures include compounds of the general formula

| | |
|---|---|
| C_{y}-Y-G-F-X-W-G-Z-C_{yy} | (SEQ ID NO: 28) |

as defined above, wherein C_{y} and C_{yy} are residues forming a lactam bridge, such as terminal amino acids, aspartic acid (D), glutamic acid (E), lysine (K), ornithine (0), or analogues thereof comprising no more than 12 carbon atoms.

In a preferred embodiment according to the present invention, C_{y} is glutamic acid, aspartic, or a structural or functional analogue thereof when C_{yy} is lysine, ornithine, or a structural or functional analogue thereof; or C_{y} is lysine, ornithine, or a structural or functional analogue thereof when C_{yy} is glutamic acid, aspartic, or a structural or functional analogue thereof.

Examples of structural or functional analogues of lysine and ornithine include any optical isomers of lysine or ornithine, and structural and/or functional analogues thereof, that preferably comprise at least one amino group or substituted amino group or other nitrogen-containing group that has or can through protonation gain a positive charge.

Further, examples of structural or functional analogues of lysine can be characterized by the presence of two amino groups or substituted amino groups (such as the monoethylamino group) or equivalents thereof, as well as the presence of at least one carboxyl group or its equivalent (such as an acyl chloride group), in the molecule.

Structural or functional analogues of lysine and ornithine may be selected e.g. from the group of ornithine, lysine, any C-methylated analogues of ornithine or lysine, any C,C′-dimethylated analogues of ornithine or lysine, 2,4-diaminobutanoic acid, 2,7-diaminoheptanoic acid, 2,8-diaminooctanoic acid, 2,4-diamino-3-methylbutanoic acid, 2,7-diamino-3-methyl-heptanoic acid, 2,8-diamino-3-methyl-octanoic acid, 2,4-diamino-2-methylbutanoic acid, 2,7-diamino-2-methyl-heptanoic acid, 2,8-diamino-2-methyl-octanoic acid, 2,4-diamino-2-methylbutanoic acid, 2,7-diamino-2-methyl-heptanoic acid, 2,8-diamino-2-methyl-octanoic acid, 2,4-diamino-2,3-dimethyl-butanoic acid, 2,4-diamino-3-methylbutanoic acid, 2,7-diamino-3-methyl-heptanoic acid, 2,8-diamino-3-methyl-octanoic acid, 2,4-diamino-2-methylbutanoic acid, 2,7-diamino-2-methyl-heptanoic acid, 2,8-diamino-2-methyl-octanoic acid, 2,4-diamino-2-methylbutanoic acid, 2,7-diamino-2-methyl-heptanoic acid, 2,8-diamino-2-methyl-octanoic acid, 2,7-diamino-3-ethyl-heptanoic acid, 2,8-diamino-3-ethyl-octanoic acid, 2,7-diamino-2-ethyl-heptanoic acid, 2,8-diamino-2-ethyl-octanoic acid, 2,4-diamino-2-ethylbutanoic acid, 2,7-diamino-2-ethyl-heptanoic acid, 2,8-diamino-2-ethyl-octanoic acid, 2,7-diamino-3-ethyl-heptanoic acid, 2,8-diamino-3-ethyl-octanoic acid, 2,4-diamino-3-ethyl-1-cyclohexanoic acid, 2,5-diamino-1-cyclohexanoic acid, 2,8-diamino-3-ethyl-1-cyclooctanoic acid, 2,4-diamino-3-ethyl-1-cycloheptanoic acid, or 2,5-diamino-1-cyclononanoic acid.

In a preferred embodiment of the present invention, C_{y} is selected from the group consisting of a diamino acid (such as lysine, ornithine, or a structural or functional analogue thereof) and an N-terminal D-amino acid, when C_{yy} is selected from the group consisting of an L-amino dicarboxylic acid (such as glutamic acid, aspartic acid, or a structural or functional analogue thereof).

“N-terminal D-amino acid” refers to an amino acid, which is a D-stereoisomer, located at the amino terminal end of the peptide chain, and bonded by its terminal amino group to a lactam bridge that may be of the “head to side chain”-type.

In a further preferred embodiment of the present invention, the lactam is a “head to side chain” lactam, wherein C_{y} is a D-amino acid, such as D-alanine (a), and C_{yy} is an L-amino dicarboxylic acid, such as glutamic acid, which may be coupled via its C-terminal carboxyl with another moiety, such as a linker.

Preferred linear or lactam-bridged targeting units according to the present invention are aYGFVWGEE (SEQ ID NO. 17), aYGFVWGQE (SEQ ID NO. 18), aYGFLWGQE (SEQ ID NO. 19), aYGFLWGEE (SEQ ID NO. 20), aYGFAWGQE (SEQ ID NO. 21), aYGFAWGEE (SEQ ID NO. 22), aYGFIWGQE (SEQ ID NO. 23), aYGFIWGEE (SEQ ID NO. 24), where a is D-alanine.

Targeting Agents According to the Present Invention

It has now also been found that targeting agents comprising at least one tumor targeting unit according to the present invention, and at least one effector unit (EU), target to and exhibit selective binding to cancer cells and cancer tissues.

The tumor targeting agents according to the present invention may optionally comprise unit(s) such as linkers, solubility modifiers, stabilizers, charge modifiers, spacers, lysis or reaction or reactivity modifiers, internalizing units or internalization enhancers or membrane interaction units or other local route, attachment, binding or distribution affecting units or other related units. Such additional units of the tumor targeting agents according to the present invention may be coupled to each other by any means suitable for that purpose.

Many possibilities are known to those skilled in the art for linking structures, molecules and groups of the types in question or of related types, to each other. The various units may be linked either directly or with the aid of one or more identical, similar and/or different linker units. The tumor targeting agents of the invention may have different structures such as any of the non-limiting types schematically shown below:

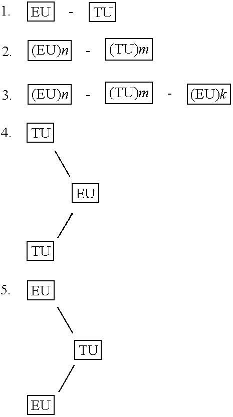

where EU indicates “effector unit” and TU indicates “targeting unit” and n, m and k are independently any integers except 0.

In a targeting agent according to the present invention, as in many other medicinal and other substances, it may be wise to include spacers or linkers, such as amino acids and their analogues, such as long-chain omega-amino acids, to prevent the targeting units from being ‘disturbed’ sterically or electronically, or otherwise hindered or ‘hidden’, by effector units or other units of the targeting agent.

In targeting agents according to the present invention, it may be useful for increased activity to use dendrimeric or cyclic structures for example to provide a possibility to incorporate multiple effector units or additional units per targeting unit.

Preferred targeting agents according to the present invention comprise a structure EU-TU-OU, TU-EU-OU or TU-OU-EU, wherein TU is a targeting unit according to the present invention as defined above; and EU and OU are effector or optional units (OU) selected from the group consisting of:

effector units, linker units, solubility modifier units, stabilizer units, charge modifier units, spacer units, lysis and/or reaction and/or reactivity modifier units, internalizing and/or internalization enhancer and/or membrane interaction units and/or other local route and/or local attachment/local binding and/or distribution affecting units, adsorption enhancer units, and other related units; and

peptide sequences and other structures comprising at least one such unit; and

peptide sequences comprising no more than 20, preferably no more than 12, more preferably no more than 6, natural and/or unnatural amino acids; and

natural and unnatural amino acids comprising no more than 25 non-hydrogen atoms and an unlimited number of hydrogen atoms;

as well as salts, esters, derivatives and analogues thereof.

Effector Units

For the purposes of this invention, the term “effector unit” (EU) means molecules or radicals or other chemical entities or large particles such as colloidal particles and their like; liposomes, nanoparticles or microgranules and their like. Suitable effector units may also comprise nanodevices or nanochips or their like; or a combination of any of the aforementioned, and optionally chemical structures for the attachment of the constituents of the effector unit to each other or to other parts of the targeting agents. Effector units may also contain moieties that modify the stability or solubility of the effector units.

Preferred effects provided by the effector units according to the pre-sent invention are therapeutic (biological, chemical or physical) effects on the targeted tumor; properties that enable the detection or imaging of tumors or tumor cells for diagnostic purposes; or binding abilities that relate to the use of the targeting agents in different applications.

A preferred (biological) activity of the effector units according to the present invention is a therapeutic effect. Examples of such therapeutic activities, are for example, cytotoxicity, cytostatic effects, ability to cause differentiation of cells or to increase their degree of differentiation or to cause phenotypic changes or metabolic changes, chemotactic activities, immunomodulating activities, pain relieving activities, radioactivity, ability to affect the cell cycle, ability to cause apoptosis, hormonal activities, enzymatic activities, ability to transfect cells, gene transferring activities, ability to mediate “knock-out” of one or more genes, ability to cause gene replacements or “knock-in”, ability to decrease, inhibit or block gene or protein expression, antiangiogenic activities, ability to collect heat or other energy from external radiation or electric or magnetic fields, ability to affect transcription, translation or replication of the cell's genetic information or external related information, and to affect post-transcriptional or post-translational events, and so on.

Other preferred therapeutic uses enabled by the effector units according to the present invention may be the administration of an enzyme capable of hydrolyzing for example an ester bond or other bonds or the administration of a targeted enzyme according to the present invention.

One further preferred therapeutic use enabled by the effector units according to the present invention may be the use of neutron capture therapy-active (NCT-active) substances as effector units. By NCT-active substances is meant any substance that by virtue of its ability to become radioactive by capture of slow neutrons can be used for neutron capture therapy (i.e. that emits radiation after having captured slow neutrons).

Examples of preferred functions of the effector units according to the present invention suitable for detection are radioactivity, paramagnetism, ferromagnetism, ferrimagnetism, or any type of magnetism, or ability to be detected by NMR spectroscopy, or ability to be detected by EPR (ESR) spectroscopy, or suitability for PET imaging (PET-active substances) and/or SPECT imaging (SPECT-active substances). By PET-active substances is meant any substance that can be used for positron emission tomography (PET). By SPECT-active substances is meant any substance that can be used for single photon emission computer tomography (SPECT) by virtue of its ability to emit photons.

Other examples of preferred properties of the effector units according to the present invention suitable for direct or indirect detection include presence of an immunogenic structure, or the presence of an antibody or antibody fragment or antibody-type structure, or the presence of a gold particle, or the presence of biotin or avidin or other protein, and/or luminescent and/or fluorescent and/or phosphorescent activity or the ability to enhance detection of tumors, tumor cells, endothelial cells and metastases in electron microscopy, light microscopy (UV and/or visible light), infrared microscopy, atomic force microscopy or tunneling microscopy, and so on.

Preferred detectable substances according to the present invention may comprise a chelator; a complexed metal such as a rare earth metal, a paramagnetic metal, a fluorescent metal (e.g. Eu, Tb or Ho), a radioactive metal, a PET-active substance or a SPECT-active substance; an enriched isotope; radioactive material such as beta-emittor or alpha emittor; a paramagnetic substance; an affinity label; a fluorescent label (e.g. fluorescein or rhodamine) or a luminescent label.

Preferred binding abilities of an effector unit according to the pre-sent invention include, for example:

a) ability to bind metal ion(s) e.g. by chelation,

b) ability to bind a cytotoxic, apoptotic or metabolism affecting substance or a substance capable of being converted in situ into such a substance,

c) ability to bind to a substance or structure such as a histidine tag or other tag,

d) ability to bind to an enzyme or a modified enzyme,

e) ability to bind to biotin or analogues thereof,

f) ability to bind to avidin or analogues thereof,

g) ability to bind to integrins or other substances involved in cell adhesion, migration, or intracellular signaling,

h) ability to bind to phages,

i) ability to bind to lymphocytes or other blood cells,

j) ability to bind to any preselected material by virtue of the presence of antibodies or structures selected by biopanning or by other methods,

k) ability to bind to material used for signal production or amplification,

l) ability to bind to therapeutic substances.

Such binding may be the result of e.g. chelation, formation of covalent bonds, antibody-antigen-type affinity, ion pair or ion associate formation, specific interactions of the avidin-biotin-type, or the result of any type or mode of binding or affinity.

In one preferred embodiment according to the present invention metals for chelation are fluorescent metals such as europium (Eu), terbium (Tb) or holmium (Ho).

One or more of the effector units or parts of them may also be a part of the targeting units themselves. Thus, the effector unit may for example be one or more atoms or nuclei of the targeting unit, such as radioactive atoms (such as carbon-13, carbon-11, carbon-14, fluorine-18 or tritium) or atoms that can be made radioactive (e.g. boron-10), or paramagnetic atoms (e.g. gadolinium (Gd) or iron) or atoms that are easily detected by MRI or NMR spectroscopy. Further examples of effector units are, for example, boron-comprising structures such as carborane-type structures.

The effector units may be linked to the targeting units by any type of bond or structure or any combinations of them that are strong enough so that most, or preferably all or essentially all of the effector units of the targeting agents remain linked to the targeting units during the essential (necessary) targeting process, e.g. in a human or animal subject or in a biological sample under study or treatment.

The effector units or parts of them may remain linked to the targeting units, or they may be partly or completely hydrolyzed or otherwise disintegrated from the latter, either by a spontaneous chemical reaction or equilibrium or by a spontaneous enzymatic process or other biological process, or as a result of an intentional operation or procedure such as the administration of hydrolytic enzymes or other chemical substances. It is also possible that the enzymatic process or other reaction is caused or enhanced by the administration of a targeted substance such as an enzyme in accordance with the present invention.

One possibility is that the effector units or parts thereof are hydrolyzed from the targeting agent or hydrolyzed into smaller units by the effect of one or more of the various hydrolytic enzymes present in tumors (e.g., intracellularly, in the cell membrane or in the extracellular matrix) or in their near vicinity.

Taking into account that the targeting according to the present invention may be very rapid, even non-specific hydrolysis that occurs every-where in the body may be acceptable and usable for hydrolyzing one or more effector unit(s) intentionally, since such hydrolysis may in suitable cases (e.g., steric hindrance, or even without any such hindering effects) be so slow that the targeting agents are safely targeted in spite of the presence of hydrolytic enzymes of the body, as those skilled in the art very well understand. The formation of insoluble products or products rapidly absorbed into cells or bound to their surfaces after hydrolysis may also be beneficial for the targeted effector units or their fragments etc. to remain in the tumors or their closest vicinity.

In one preferred embodiment of the invention, the effector units may comprise structures, features, fragments, molecules or the like that make possible, cause directly or indirectly, an “amplification” of the therapeutic or other effect, of signal detection, of the binding of preselected substances, including biological material, molecules, ions, microbes or cells.

Such “amplification” may, for example, be based on one or more of the following non-limiting types:

the binding, by the effector units, of other materials that can further bind other substances (for example, antibodies, fluorescent antibodies, other “labeled” substances, substances such as avidin), preferably so that several molecules or “units” of the further materials can be bound per each effector unit;

the effector units comprise more than one entity capable of binding e.g. a protein, thus making direct amplification possible;

amplification in more than one steps.

Preferred effector units according to the present invention may be selected from the following group:

cytostatic or cytotoxic agents

apoptosis causing or enhancing agents

enzymes or enzyme inhibitors

antimetabolites

agents capable of disturbing membrane functions

radioactive or paramagnetic substances

substances comprising one or more metal ions

substances comprising boron, gadolinium, litium

substances suitable for neutron capture therapy, e.g. boron or carborane

labeled substances

intercalators and substances comprising them

oxidants or reducing agents

amino acids, nucleic acids, nucleotides and their analogues (including aptamers, peptide nucleic acids (PNA) and anti-sense oligonucleotides)

metal chelates or chelating agents.

In a preferred embodiment of the invention, the effector unit may comprise cytostatic/cytotoxic agents such as 5-fluorouracil, leucovorin, oxaliplatin, irinotecan, or polyketidic antracyclines including doxorubicin and daunorubicin.

In another preferred embodiment of the invention, the effector unit comprises radiation emitting substances such as alpha-emittors, beta-emittors, gamma-emittors or NCT-active substances.

In further preferred embodiments of the invention, the effector units may comprise copper chelates such as trans-bis(salicylaldoximato)copper(II) and its analogues, or platinum compounds such as cisplatin and carboplatin.

More specifically, for the treatment of colon cancer, in combination with conventional therapeutics, such as platinum compounds, the following effector units or their structural or functional analogs may be used: mitosis inhibitors/taxanes such as paclitaxel or docetaxel; anti-metabolites such as gemsitabine or metotrexate; vinca alkaloids such as vinorelbine or vincristine; alkylating agents such as isophosphamide or cyclophosphamide; antibiotics such as bleomycine or mitomycine; or topoisomerase inhibitors such as irinotecane or topotecane.

Different types of structures, substances and groups are known that can be used to cause or enhance e.g., internalization into cells, including for example RQIKIWFQNRRMKWKK (SEQ ID NO: 29); Penetratin (Prochiantz, 1996, Curr. Op. Neurobiol., 6: 629-634), as well as stearyl derivatives (Promega Notes Magazine, 2000).

As an apoptosis-inducing structure, for example, the peptide sequence KLAKLAK (SEQ ID NO: 30) that has been reported to interact with mitochondrial membranes inside cells, can be included (Ellerby et al. 1999, Nat. Med., 9: 1032-1038).

For use in embodiments of the present invention that include cell sorting or any related applications, the targeting units and agents of the invention can, for example, be used

a) coupled or connected to magnetic particles,

b) adsorbed, coupled, linked or connected to plastic, glass or other solid, porous, fibrous material-type or other surface(s) and their like,

c) adsorbed, covalently bonded or otherwise linked, coupled or connected into or onto one or more substance(s) or material(s) that can be used in columns or related systems

d) adsorbed, covalently bonded or otherwise linked, coupled or connected into or onto one or more substance(s) or material(s) that can be precipitated, centrifuged or otherwise separated or removed.

Optional Units (Ou) of the Targeting Agents According to the Present Invention

The targeting agents and targeting units of the present invention may optionally comprise further units, such as:

linker units for coupling targeting units, effector units, or other optional units of the present invention to each other;

solubility modifying units for modifying the solubility of the targeting agents or their hydrolysis products;

stabilizer units for stabilizing the structure of the targeting units or agents during synthesis, modification, processing, storage or use in vivo or in vitro; charge modifying units for modifying the electrical charges of the targeting units or agents or their starting materials;

spacer units for increasing the distance between specific units of the targeting agents or their starting materials, for releasing or decreasing steric hindrance or structural strain of the products or their starting materials;

reactivity modifier units;

internalizing units or enhancer units for enhancing targeting or uptake of the targeting agents;

adsorption enhancer units, such as fat soluble or water soluble structures that for example enhance absorption of the targeting agents in vivo; or other related units.

A large number of suitable linker units are known in the art. Examples of suitable linkers are:
1. for linking units that comprise amino groups: cyclic anhydrides, dicarboxylic or multivalent, optionally activated or derivatized, carboxylic acids, compounds with two or more reactive halogens or compounds with at least one reactive halogen atom and at least one carboxyl group;
2. for linking units that comprise carboxyl groups or derivatives thereof: compounds with at least two similar or different groups such as amino, substituted amino, hydroxyl, —NHNH₂ or substituted forms thereof, other known groups for the purpose (activators may be used);
3. for linking an amino group and a carboxyl group: for example amino acids or their activated or protected forms or derivatives;
4. for linking a formyl group or a keto group to another group: a compound comprising e.g. at least one —N—NH₂ or —O—NH₂ or ═N—NH₂ group or their like;
5. for linking several amino-comprising units: polycarboxylic substances such as EDTA, DTPA or polycarboxylic acids, or anhydrides, esters or acyl halides thereof;
6. for linking a substance comprising an amino group to a substance comprising either a formyl group or a carboxyl group: hydrazinocarboxylic acids or their like, preferably so that the hydrazino moiety or the carboxyl group is protected or activated, such as 4-(FMOC-hydrazino)benzoic acid;
7. for linking an organic structure to a metal ion: substances that can be coupled to the organic structure (e.g. by virtue of their COOH groups or their NH2 groups) or that are integral parts of it, and that in addition comprise a polycarboxylic part, for example an EDTA- or DTPA-like structure, peptides comprising several histidines or their like, peptides comprising several cysteines or other moieties comprising an —SH group each, or other chelating agents that comprise functional groups that can be used to link them to the organic structure.

A large variety of the above substances and of other types of suitable linking agents is known in the art.

A large number of suitable solubility modifier units are known in the art. Suitable solubility modifier units may comprise, for example:

for increasing aqueous solubility: Molecules comprising SO₃^{(-), OSO}₃(−), COOH, COO^{(-), NH}₂, NH₃⁽⁺⁾, OH, phosphate groups, guanidino or amidino groups or other ionic or ionizable groups or sugar-type structures;

for increasing fat solubility or solubility in organic solvents: Units comprising (long) aliphatic branched or non-branched alkyl or alkenyl groups, cyclic non-aromatic groups such as the cyclohexyl group, aromatic rings or steroidal structures.

One especially preferred aqueous-solubility enhancing unit comprises at least one unit according to Formula I:
—(CH₂)ₘ—O— (I)

where m is an integer of value 1 to 4;

or at least one unit according to Formula II:
-(A)ₛ-Y (II)

where (A)ₛ is a spacer group wherein each A is independently CR₁R₂,

each R₁ and R₂ is independently selected from the group of hydrogen, hydroxyl, C₁₋₃ alkyl and C₁₋₃ hydroxyalkyl,

s is an integer of value 0 to 5, and

Y is selected from COOH, CONH₂, NH₂ and guanyl;

or at least one unit according to Formula III:

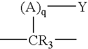

where (A)_{q} is a spacer group wherein each A is independently CR₁R₂,

q is an integer of value 1 to 5,

each R₁, R₂ and R₃ is independently selected from the group of hydrogen, hydroxyl, C₁₋₃ alkyl and C₁₋₃ hydroxyalkyl, and

Y is selected from from the group of COOH, CONH₂, NH₂ and guanyl.

Useful aqueous solubility enhancing units may comprise at least one, preferably at least three, more preferably at least six and most preferably at least nine units according to Formula I, or at least one unit according to Formula II, or they may comprise at least one and more preferably at least four units according to Formula III.

Specific examples of targeting agents comprising aqueous-solubility enhancing units are further described in the examples. Such targeting agents have valuable characteristics and excellent properties for diagnostic and therapeutic uses.

A large number of units known in the art can be used as stabilizer units, e.g. bulky structures (such as tert-butyl groups, naphthyl and adamantyl and related radicals etc.) for increasing steric hindrance, and D-amino acids and other unnatural amino acids (including beta-amino acids, omega-amino acids, amino acids with very large side chains etc.) for preventing or hindering enzymatic hydrolysis.

Units comprising positive, negative or both types of charges can be used as charge modifier units, as can also structures that are converted or can be converted into units with positive, negative or both types of charges.

Spacer units may be very important, and the need to use such units depends on the other components of the structure (e.g. the type of biologically active agents used, and their mechanisms of action) and the synthetic procedures used.

Suitable spacer units may include for example long aliphatic chains or sugar-type structures (to avoid too high lipophilicity), or large rings. Suitable compounds are available in the art. One preferred group of spacer units are omega-amino acids with long chains. Such compounds can also be used (simultaneously) as linker units between an amino-comprising unit and a carboxyl-comprising unit. Many such compounds are commercially available, both as such and in the forms of various protected derivatives.

Units that are susceptible to hydrolysis (either spontaneous chemical hydrolysis or enzymatic hydrolysis by the body's own enzymes or enzymes administered to the patient) may be very advantageous in cases where it is desired that the effector units are liberated from the targeting agents e.g. for internalization, intra- or extracellular DNA or receptor binding. Suitable units for this purpose include, for example, structures comprising one or more ester or acetal functionality. Various proteases may be used for the purposes mentioned. Many groups used for making pro-drugs may be suitable for the purpose of increasing or causing hydrolysis, lytic reactions or other decomposition processes.

The effector units, the targeting units and the optional units according to the present invention may simultaneously serve more than one function. Thus, for example, a targeting unit may simultaneously be an effector unit or comprise several effector units; a spacer unit may simultaneously be a linker unit or a charge modifier unit or both; a stabilizer unit may be an effector unit with properties different from those of another effector unit, and so on. An effector unit may, for example, have several similar or even completely different functions.

In one preferred embodiment of the invention, the tumor targeting agents comprise more than one different effector units. In that case, the effector units may be, for example, diagnostic and therapeutic units. Thus, for ex-ample, it is preferred to use, for boron neutron capture therapy, such agents whose effector units, in addition to comprising boron atoms, also can be detected or quantified in the patient in vivo after administration of the agent, in order to be able to ascertain that the agent has accumulated adequately in the tumor to be treated, or to optimize the timing of the neutron treatment, and so on. This goal may be achieved e.g. by using such a targeting agents according to the invention that comprise an effector unit comprising boron atoms (preferably isotope-enriched boron) and groups detectable e.g. by PET, SPECT or NMRI. Likewise, the presence of more than one type of therapeutically useful effector units may also be preferred. In addition, the targeting units and targeting agents may, if desired, be used in combination with one or more “classical” or other tumor therapeutic modalities such as surgery, chemotherapy, other targeting modalities, radiotherapy, immunotherapy etc.

Preparation of Targeting Units and Agents According to the Present Invention

The targeting units according to the present invention are preferably synthetic peptides. Peptides can be synthesized by a large variety of well-known techniques, such as solid-phase methods (FMOC-, BOC-, and other protection schemes, various resin types), solution methods (FMOC, BOC and other variants) and combinations of these. Automated apparatuses/devices for the purpose are available commercially, as are also routine synthesis and purification services. All of these approaches are very well known to those skilled in the art.

As known in the art, it is often advisable, important and/or necessary to use one or more protecting groups, a large variety of which are known in the art, such as FMOC, BOC, and trityl groups and other protecting groups mentioned in the Examples. Protecting groups are often used for protecting amino, carboxyl, hydroxyl, guanyl and —SH groups, and for any reactive groups/functions.

As those skilled in the art well know, activation often involves carboxyl function activation and/or activation of amino groups.

Protection may also be orthogonal and/or semi/quasi/pseudo- orthogonal. Protecting and activating groups, substances and their uses are exemplified in the Examples and are described in the references cited herein, and are also described in a large number of books and other sources of information commonly known in the art.

Resins for solid-phase synthesis are also well known in the art, and are described in the Examples and in the above-cited references.

Cyclic peptides are usually especially stable in biological milieu, and are thus preferred. Cyclic structures according to the present invention may be synthesized by methods based on the use of orthogonally protected amino acids, as described in e.g., International Patent Publication WO 2004/031218, incorporated herein by reference.

The targeting units and agents according to the present invention may also be prepared as fusion proteins or by other suitable recombinant DNA methods known in the art. Such an approach for preparing the peptides according to the present invention is preferred especially when the effector units and/or other optional units are peptides or proteins. One example of a useful protein effector unit is glutathione-S-transferase (GST).

Advantages of the Targeting Units and Targeting Agents of the Invention

There are acknowledged problems related to peptides intended for diagnostic or therapeutic use. One of these problems stems from the length of the sequence: The longer it grows, the more difficult the synthesis of the desired product becomes, especially if there are other synthetic problems such as the presence of difficult residues that require protection-deprotection or cause side reactions.

As compared to known peptides that contain long and difficult-to-make sequences with problematic amino acid residues, the peptides of the present invention are clearly superior. The targeting units of this invention can be synthesized easily and reliably. An advantage as compared to many prior art peptides is that the targeting units and motifs of this invention do not need to comprise the problematic basic amino acids lysine and histidine, both of which may cause serious side-reactions in peptide synthesis, and, due to which the yield of the desired product might be lowered radically or even the product might be impossible to obtain in adequate amounts or with adequate quality.

Because of their smaller size and thus drastically less steps in the synthesis, the peptides of the present invention are much easier and cheaper to produce than most targeting peptides of the prior art.

As histidine is not needed in the products of the present invention, the risk of racemization is of no concern. It is a great advantage not only for the economic synthesis of the products of the present invention but also for the purification and analysis and quality control that any racemization of histidine is outside consideration. It also makes any administration to humans and animals safer and more straightforward.

Because of the smaller size of the targeting units, overall costs are drastically reduced and better products can be obtained and in greater amounts, due to easier and more reliable purification. Furthermore, the reliability of the purification is much better, giving less concern of toxic remainders and of fatal or otherwise serious side effects in therapeutic and diagnostic applications.

The targeting units of the present invention are also highly advantageous due to their specificity, non-toxicity and non-immunogenicity.

In the solid phase synthesis of targeting agents according to the present invention, the effector units and optional additional units may be linked to the targeting peptide when it is still connected to the resin, without the risk that the removal of the protecting groups will cause destruction of the effector or optional units. Similar advantages apply to solution syntheses.

Another important advantage of the present invention and the products, methods and uses according to it is the highly selective and potent targeting of the products.

As compared to targeted therapy using antibodies or antibody fragments, the products and methods of in the present invention are highly advantageous because of several reasons. Potential immunological and related risks are obvious in the case of large biomolecules. Allergic reactions are of great concern with such products, in contrast to small synthetic molecules such as the targeting agents, units and motifs of the present invention.

As compared to targeting antibodies or antibody fragments, the products and methods described in the present invention are highly advantageous because their structure can be easily modified if needed or desired. Specific amino acids such as histidine, and threonine can be omitted, if desired, and very few functional groups are necessary. On the other hand, it is possible, without disturbing the targeting effect, to include various different structural units, to obtain specific desired properties that are of special value in specific applications.

Use of the Targeting Agents According to the Present Invention

The targeting units and targeting agents according to the present invention are useful in cancer diagnostics and therapy, as they selectively tar-get to tumors, especially to colon tumors in vivo, as shown in the Examples. The effector unit may be chosen according to the desired effect, detection or therapy. The desired effect may also be achieved by including the effector in the targeting unit as such. For use in radionuclide therapy the targeting unit itself may be e.g., radioactively labeled.

The present invention also relates to diagnostic compositions comprising an effective amount of at least one targeting agent according to the present invention. A diagnostically effective amount of the targeting agents according to the present invention may range from 1 femtomol to 10 mmols, depending for example on the effector unit of choice. In addition to the targeting agent, a diagnostic composition according to the present invention may, optionally, comprise carriers, solvents, vehicles, suspending agents, labeling agents and other additives commonly used in diagnostic compositions. Such diagnostic compositions are useful in diagnosing tumors, tumor cells and metastasis, especially tumors of the colon, more specifically colon primary tumors and metastases, in animals as well as in human subjects.

A diagnostic composition according to the present invention may be formulated as a liquid, gel or solid formulation or as an inhalation formulation, etc., preferably as an aqueous liquid, containing a targeting agent according to the present invention in a concentration ranging from about 1×10⁻¹⁰ mg/l mg/l to 25×10⁴ mg/I. The compositions may further comprise stabilizing agents, detergents, such as polysorbates, as well as other additives. The concentrations of these components may vary significantly depending on the formulation used. The diagnostic compositions may be used in vivo or in vitro.

The targeting agents and targeting units according to the present invention are useful in veterinary and human therapy.

The present invention also includes the use of the targeting agents and targeting units for the manufacture of pharmaceutical compositions for the treatment of cancer.

The present invention also relates to pharmaceutical compositions comprising a therapeutically effective amount of at least one targeting agent according to the present invention. The pharmaceutical compositions may be used to treat, prevent or ameliorate cancer diseases, by administering a therapeutically effective dose of the pharmaceutical composition comprising targeting agents or targeting units according to the present invention or therapeutically acceptable salts, esters or other derivatives thereof. The compositions may also include different combinations of targeting agents and targeting units together with labeling agents, imaging agents, drugs and other additives.

A therapeutically effective amount of a targeting agent according to the present invention may vary depending on the formulation of the pharmaceutical composition. Preferably, a pharmaceutical composition according to the present invention may comprise a targeting agent in a concentration varying from about 0.00001 mg/l to 250 g/l, more preferably about 0.001 mg/l to 50 g/l, most preferably 0.01 mg/l to 20 g/l.

A pharmaceutical composition according to the present invention is useful for administration of a targeting agent according to the present invention. Pharmaceutical compositions suitable for per oral use, for intravenous or local injection, or infusion, or inhalation are particularly preferred. The pharmaceutical compositions may be used in vivo or ex vivo.

The preparations may be lyophilized and reconstituted before administration or may be stored for example as a solutions, suspensions, suspension-solutions etc. ready for administration or in any form or shape in general, including powders, concentrates, frozen liquids, and any other types. They may also consist of separate entities to be mixed and, possibly, otherwise handled and/or treated etc. before use. Liquid formulations provide the advantage that they can be administered without reconstitution. The pH of the solution product is in the range of about 1 to about 12, preferably close to physiological pH. The osmolality of the solution can be adjusted to a preferred value using for example sodium chloride and/or sugars, polyols and/or amino acids and/or similar components. The compositions may further comprise pharmaceutically acceptable excipients and/or stabilizers, such as albumin, sugars and various polyols, as well as any acceptable additives, or other active ingredients such as chemotherapeutic agents.

The present invention also relates to methods for treating cancer, especially solid tumors by administering to an animal subject, including a human patient, in need of such treatment a therapeutically efficient amount of a pharmaceutical composition according to the present invention.

Therapeutic doses may be determined empirically by testing the targeting agents and targeting units in available in vitro or in vivo test systems. Suitable therapeutically effective dosage may then be estimated from these experiments.

For oral administration it is important that the targeting units and targeting agents are stable and adequately absorbed from the intestinal tract.

The pharmaceutical compositions according to the present invention may be administered systemically, non-systemically, locally or topically, par-enterally as well as non-parenterally, e.g. subcutaneously, intravenously, in-tramuscularly, per orally, intranasally, by pulmonary aerosol or powder, by injection or infusion into a specific organ or region, buccally, intracranically or intraperitoneally etc.

Amounts and regimens for the administration of the tumor targeting agents according to the present invention can be determined readily by those with ordinary skill in the clinical art of treating cancer. Generally, the dosage will vary depending upon considerations such as: type of targeting agent employed; age; health; medical conditions being treated; kind of concurrent treatment, if any; frequency of treatment and the nature of the effect desired; gender; duration of the symptoms; and, counterindications, if any, and other variables to be adjusted by the individual physician. Preferred doses for ad-ministration to human patients of targeting units or agents according to the present invention may vary from about 1×10⁻⁹ mg to about 40 mg per kg of body weight as a bolus or repeatedly, e.g., as daily doses.

The targeting units and targeting agents and pharmaceutical compositions of the present invention may also be used as targeting devices for delivery of DNA or RNA or structural and functional analogues thereof, such as phosphorothioates, or peptide nucleic acids (PNA) into tumors and their metastases or to isolated cells and organs in vitro; i.e. as tools for gene therapy both in vivo and in vitro. In such cases the targeting agents or targeting units may be parts of viral capsids or envelopes, of liposomes or other “containers” of DNA/RNA or related substances, or may be directly coupled to the DNA/RNA or other molecules mentioned above. An especially preferred embodiment of the present invention is a targeting agent comprising a TU as an amino acid chain or its structural or functional analogue, and an EU as a PNA or its analogue, linked together via a peptide bond, as one contiguous molecule. Such a targeting agent may be used for intracellular delivery of small interfering RNA (siRNA; in this case “siPNA”) for gene product-specific inhibition (silencing) of gene expression.

The targeting units and agents according to the present invention may also be modified to improve stability, e.g. lengthen the biological half-life thereof by increasing the retention or stability of the targeting unit in the desired environment such as mammalian circulation. Such properties are achieved by standard pharmaceutical formulation chemistry tools and include introduction of non-hydrolysable bonds, glycosylation, pegulation as well as mixing with pharmaceutically acceptable diluents, adjuvants, carriers or vehicles well know to a person skilled in the art. The targeting units may also be chemically modified to decrease in vivo proteolytic digestion thereof by methods known in the art.

The present invention also includes kits and components of kits for diagnosing, detecting or analyzing cancer or cancer cells in vivo and in vitro. Such kits comprise at least one targeting agent or targeting unit of this invention together with diagnostic entities enabling detection. The kit may comprise for example a targeting agent or a targeting unit coupled to a unit for detection by e.g. immunological methods, radiation or enzymatic methods or other methods known in the art.

Further, the targeting units and agents of this invention as well as the targeting motifs and sequences can be used as lead compounds to design peptidomimetics for any of the purposes described above.

Yet further, the targeting units and agents as well as the targeting motifs and sequences of the present invention, as such or as coupled to other materials, can be used for the isolation, purification and identification of the cells, molecules and related biological targets.

The following examples are given to further illustrate preferred embodiments of the present invention, but are not intended to limit the scope of the invention. It will be obvious to a person skilled in the art, as the technology advances, that the inventive concept can be implemented in various ways. The invention and its embodiments are not limited to the examples described above but may vary within the scope of the claims.

### EXAMPLES

### Example 1

General screening method for bio-panning of patient samples

Phage display library. Standard procedures according to Smith and Scott (ibid.) were used. Phage display library used for screening of clinical samples was cloned in fUSE5 vector and was of the cyclic structure CX7C. The E. coli strain K91kan was used as host for phage amplification.

Phage display on clinical tumor samples. Tissue samples were surgically removed from lung metastases of colorectal cancer patients. Part of the sample was taken for pathological examination, rest was placed in ice cold DMEM-PI (Dulbecco's medium containing protease inhibitors PI; 10 mM PMSF (Phenyl-methyl-sulphonyl-fluoride), Aprotinin (10 mg/ml) Leupeptin (10 mg/ml)). Tissue samples were minced with a razor blade in a small cell culture plate in 1 ml of DMEM containing protease inhibitors. The samples were transferred to an eppendorf tube and washed with 1 ml DMEM-PI.

Samples were centrifuged at 5000 rpm for 4 min and were then incubated with 1010 transducing units of phage in 1 ml DMEM-PI at 25° C. for 15 min. After this the samples were washed three times with DMEM-PI containing 1% BSA (bovine serum albumin).

1 ml K91kan bacteria, OD600: 1-1.5, in LB containing 100 μg/ml kanamycin (kan) were infected with the washed pellet containing selectively attached phage particles at 25° C. for 25 min. After infection volume was increased to 5 ml with LB containing 100 μg/ml kan.

Then infected bacteria were plated on LB agar plates containing 40 μg/ml tetracycline (tet) as follows: Three parallel plates of three dilutions (1:250,1:2500, 1:25000) and the rest of the above K91kan culture in 300 μl aliquots. The plates were incubated overnight at +37° C.

The following day colonies were picked from the LBtet plates into 96-well micro-plates for sequencing of the phage DNA. Extra clones were also stored at −20° C. for later analysis.

After picking colonies for sequencing the remaining bacterial colonies were pooled from the plates in 150 ml LB (100 μg/ml kan, 20 μg/ml tet) for further growth. The culture was grown at 37° C. for 1-1.5 h.

Then the bacteria were pelleted at 5000 rpm for 15 min. The supernatant containing amplified phages was precipitated by adding PEG to 0.04 g/ml and NaCl to 0.03 g/ml. The phages were shaken overnight at +4° C. on ice. After this the phages were pelleted by centrifugation at 10 000 rpm for 20 min at +4° C. The resulting pellet was re-suspended in TBS and then reprecipitated for 1 h at +4° C. on ice by addition of PEG/NaCl as described above. Then the phages were pelleted at 14 000 rpm for 20 min at +4° C. on ice. Finally, the pellet was re-suspended in 1 ml of TBS containing 0.02% NaN3 and stored at +4° C. For the next rounds of bio-panning of clinical samples the titre of the TBS phage stock was determined as described above.

To achieve selective enrichment of tumor targeting peptides, phage stocks prepared as described above were used three rounds of biopanning of clinical samples.

Identification of enrichment of peptides. To determine the number of sequence of selectively enriched peptides, DNA sequencing was performed on 20 to 48 colonies, representing individual phage clones, from the first round of bio-panning onwards.

First colony PCR was performed to produce DNA for sequencing: Bacterial colonies in the wells of 96-well plate were suspended to 30 μl TBS buffer and 5 μl of this were taken to PCR reaction. Next, PCR-Mix was made -PCR-Mix for one reaction is: 0.1 ml 10 mM dNTP's, 5.0 μl of template, 0.7 μl of F1-forward primer (15 μM), 0.7 μl F1-reverse primer (15 μM), 4 μl 10× Dynazyme buffer, 0.5 μl of Dynazyme polymerase (=1 U) and 29 μl of dH2O giving a final volume of 40 ml. The setting for the PCR program used was 96° C. for 5 min followed by a cycle of three steps 1) 92° C. for 30 seconds, 2) 60° C. for 30 seconds and 3) 72° C. for 1 minute. This cycle of three steps was repeated 35 times. The sequences of the primers used in PCR amplification were 5′-gCMgCTgATAAACCgATACAATTAAAgg-3′ (SEQ ID NO: 31) for F1-F and 5′-gCCC TCA TAg TTA gCg TM CgA TC-3′ for F1-R (SEQ ID NO: 32).

Prior to sequencing amplification of DNA insert of the phage clones was verified by electrophoresis. Sequencing was performed with an ALF automated DNA sequencer (AmershamPharmacia Biotech) using the F1-F and F1-R primers described above.

Peptides selectively binding to lung tumors are the following: CYGFVWGEC (SEQ ID NO: 9) and CYGFLWGQC (SEQ ID NO: 11). The enriched peptide sequences were collected from ex vivo panning round three.

### Example 2

Preparation of Synthetic Peptides

All peptide syntheses were carried out manually or by using an automated synthesis instrument (either Applied Biosystems 433A or Advanced Chem Tech 396DC). The method was solid phase peptide synthesis based on N-FMOC protection and HBTU/HOBt/DIPEA activation. The synthesis resins employed were Rink amide MBHA resin, cysteamine-2-chlorotrityl resin, 1,2-diaminoethane trityl resin or preloaded FMOC-amino acid Wang resin. In automated syntheses the standard operating procedures and reagents recommended by the manufacturers were employed.

The major reagents in these syntheses were from Applied Biosystems or from Novabiochem: Fmoc-Cys(Trt)-OH (for ‘C’), Fmoc-Tyr(tBu)-OH (for ‘Y’), Fmoc-Gly-OH (for ‘G’), Fmoc-Phe-OH (for ‘F’), Fmoc-Val-OH (for ‘V’), Fmoc-Trp(tBoc)-OH (for ‘W’), Fmoc-Glu(OtBu)-OH (for ‘E’), Fmoc-D-Ala-OH (for ‘a’) and Fmoc-Glu(O-2-Ph-i-Pr)-OH (for lactam-bridged ‘E’, i.e. for ‘E*’; the use of the asterisks herein is for indicating the amino acids connected by a lactam bridge).

The spacer unit reagent Fmoc-11-amino-3,6,9-undecanoic acid (for ‘Teg’) was purchased from University of Kuopio, Finland, and had been prepared as described previously (Boumrah, Deradji et al., Tetrahedron, 1997, 56: 6977-6992). The spacer unit reagent Fmoc-12-amino-4,7,10-trioxadodecanoic acid (for ‘TEGC’) was purchased from NeoMPS.

Linker 2-aminoethanethiol was produced via the cleavage of the cysteamine resin. Linker 1,2-diamino ethane was produced via the cleavage of the diamino ethane resin.

The thiol-reactive labeling reagent, the europium(III) chelate of p-iodoacetamidobenzyl-DTPA was prepared from 2-(4-Aminobenzyl)-diethylenetriaminepenta (t-butyl acetate) purchased from Macrocyclics, Dallas, Tex. This DTPA-Eu reagent was coupled with sulfhydryl bearing peptide compound according to Perkin Elmer's recommended procedure (PerkinElmer Wallac Ltd., Turku, Finland).

The following abbreviations are used herein:

‘Ac’ denotes: CH₃C(O) i.e. acetyl (not actinium).

‘ADGA’ (SEQ ID NO: 33) denotes: Ala-Asp-Gly-Ala (SEQ ID NO: 33).

‘AMB-DTPA-Eu’ denotes:

Eu³⁺-chelate of (p-((2-aminoethylmercapto)acetamido)benzyl)diethylenetri-amine-N,N,N′,N″,N″-pentaacetic acid coupled via primary amino group (at the aminoethyl group).

‘amide’ denotes: NH₂ group connected to carbonyl (e.g. at the C-terminus of a peptide).

‘CYGFVWGEC’ (SEQ ID NO: 9) denotes: Cys-Tyr-Gly-Phe-Val-Trp-Gly-Glu-Cys (SEQ ID NO: 9).

‘Ac-aYGFVWGEE’ (SEQ ID NO: 17) denotes: CH₃C(O)-(D-Ala)-Tyr-Gly-Phe-Val-Trp-Gly-Glu-Glu (SEQ ID NO: 17).

‘a*YGFVWGEE*’ (SEQ ID NO: 17) denotes: (D-Ala)*-Tyr-Gly-Phe-Val-Trp-Gly-Glu-Glu*; (SEQ ID NO: 17) lactam bridge between amino terminus of D-Ala* and the side chain COOH of Glu*.‘DTPA’ denotes: diethylenetriamine-N,N,N′,N″,N″-pentaacetic acid.

‘DTPA-Eu’ denotes: Eu³⁺-chelate of DTPA.

‘EAT’ denotes: 2-Aminoethanethiol, also called ethyleneaminothiol, i.e. NHCH₂CH₂SH.

‘Teg’ denotes: NH—CH₂CH₂—O—CH₂CH₂—O—CH₂CH₂—O—CH₂—C(O).

‘Teg3’ denotes: Teg-Teg-Teg, i.e. (NH—CH₂CH₂—O—CH₂CH₂—O—CH₂CH₂—O—CH₂—C(O))₃.

‘TEGC’ denotes: NH—CH₂CH₂—O—CH₂CH₂—O—CH₂CH₂—O—CH₂CH₂—C(O).

List of Reagents:

Fmoc-Cys(Trt)-OH, CAS No. 103213-32-7, Novabiochem Cat. No. 04-12-1018, Molecular Weight 585.7 g/mol.

Fmoc-Tyr(tBu)-OH, CAS No. 71989-38-3, Novabiochem Cat. No. 04-12-1037, Molecular Weight 459.6 g/mol.

Fmoc-Gly-OH , CAS No. 29022-11-5, Novabiochem Cat. No. 04-12-1001, Molecular Weight: 297.3 g/mol.

Fmoc-Phe-OH, CAS No. 35661-40-6, Novabiochem Cat. No. 04-12-1030, Molecular Weight 387.4 g/mol.

Fmoc-Val-OH, CAS No. 68858-20-8, Novabiochem Cat. No. 04-12-1039, Molecular Weight 339.4 g/mol.

Fmoc-Trp(tBoc)-OH, CAS No. 143824-78-6, Novabiochem Cat. No. 04-12-1103, Molecular Weight 526.6 g/mol.

Fmoc-Glu(OtBu)-OH, CAS No. 71989-18-9, Novabiochem Cat. No. 04-12-1020, Molecular Weight 425.5 g/mol.

Fmoc-D-Ala-OH, CAS No. 79990-15-1, Novabiochem Cat. No. 04-13-1006, Molecular Weight 311.3 g/mol.

Fmoc-Gln(Trt)-OH, CAS No. 132327-80-1, Novabiochem Cat. No. 04-12-1090, Molecular Weight 610.7 g/mol.

Fmoc-Leu-OH, CAS No. 35661-60-0, Novabiochem Cat. No. 04-12-1025, Molecular Weight 353.4 g/mol.

Fmoc-Glu(O-2-Ph-i-Pr)-OH, Novabiochem Cat. No. 04-12-1199, Molecular Weight 487.5 g/mol.

Fmoc-12-amino-4,7,10-trioxadodecanoic acid, NeoMPS Cat. No. FA19203, Molecular Weight 443.5 g/mol.

Cysteamine-2-chlorotrityl Resin, Novabiochem 01-64-0107, subst.: 1.33 mmol/g.

Rink amide MBHA Resin, Novabiochem 01-64-0107, subst.: 1.33 mmol/g.

1,2-Diaminoethanetrityl resin, Novabiochem 01-64-0081, subst.: 1.2 mmol/g.

Fmoc-Glu(OtBu)-Wang resin, Novabiochem 04-12-2052, subst.: 0.62 mmol/g.

Fmoc-Ala-OH, CAS No. 35661-39-3, Novabiochem Cat. No. 04-12-1006, Molecular Weight 311.3 g/mol.

Fmoc-Asp(OtBu)-OH, CAS No. 71989-14-5, PerSeptive Biosystems Cat. No. GEN9110211, Molecular Weight 411.5 g/mol.

General Procedures for Peptide Synthesis: Manual Aolid Phase Syntheses. Mass Spectral Measurements.

All manual synthetic procedures were carried out in a sealable glass funnel equipped with a sintered glass filter disc of porosity grade between 2 and 4, a polypropene or phenolic plastic screw cap on top (for sealing), and two PTFE key stopcocks: one beneath the filter disc (for draining) and one at sloping angle on the shoulder of the screw-capped neck (for argon gas inlet).

The funnel was loaded with the appropriate solid phase synthesis resin and solutions for each treatment, shaken effectively with the aid of a “wrist movement” bottle shaker for an appropriate period of time, followed by filtration effected with a moderate argon gas pressure.

The general procedure of one cycle of synthesis (=the addition of one amino acid unit) was as follows:

The appropriate synthesis resin loaded with approximately 0.25 mmol of FMOC-peptide (=peptide whose amino-terminal amino group was protected with the 9-fluorenylmethyloxycarbonyl group) consisting of one or more amino acid units having recommended protecting groups; approximately 0.5 g of resin (0.5 mmol/g) was treated in the way described below, each treatment step comprising shaking for one to two minutes with 10 ml of the solution or solvent indicated and filtration if not mentioned otherwise.

‘DCM’ means shaking with dichloromethane, and ‘DMF’ means shaking with N,N-dimethylformamide (DMF may be replaced by NMP, i.e., N-methylpyrrolidinone).

The steps of the treatment were:

1. DCM, shaking for 10-20 min;

2. DMF;

3. 20% (by volume) piperidine in DMF for 5 min;

4. 20% (by volume) piperidine in DMF for 10 min;

5. to 7. DMF;

8. to 10. DCM;

11. DMF;

12. DMF solution of 0.75 mmol of activated amino acid (preparation described below), shaking for 2 hours;

13. to 15. DMF;

16. to 18. DCM.

After the last treatment (18) argon gas was led through the resin for approximately 15 min and the resin was stored under argon (in the sealed reaction funnel if the synthesis was to continue with further units).

Activation of the 9-fluorenylmethyloxycarbonyl-N-protected amino acid (FMOC-amino acid) to be added to the amino acid or peptide chain on the resin was carried out, using the reagents listed below, in a separate vessel prior to treatment step no. 12. Thus, the FMOC-amino acid (0.75 mmol) was dissolved in approximately 3 ml of DMF, treated for 1 min with a solution of 0.75 mmol of HBTU dissolved in 1.5 ml of a 0.5 M solution of HOBt in DMF, and then immediately treated with 1.07 ml of a 1.4 M DIPEA solution in DMF for 5 min; exceptionally 2,4,6-trimethylpyridine was used instead of DIPEA in the case of the activation of FMOC-Cys(Trt)-OH.

The activation reagents used for activation of the FMOC-amino acid were as follows:

HBTU=2-(1H-benzotriazol-1-yl)-1,1,3,3-tetramethyluronium hexafluorophosphate, CAS No. [94790-37-1], Applied Biosystems Cat. No. 401091, molecular weight: 379.3 g/mol;

HOBt=1-Hydroxybenzotriazole, CAS No. 2592-95-2, molecular weight 135.12 g/mol, Acros Organics Cat. No. 169161000;

DIPEA=N,N-Diisopropylethylamine, CAS No. 7087-68-5, molecular weight 129.24 g/mol, Acros Organics Cat. No. 115221000.

The procedure described above is repeated in several cycles using different FMOC-amino acids, containing suitable protecting groups, to produce a “resin-bound” peptide (i.e., resinous source of an appropriate peptide). The procedure provides also a way to connect certain linker units, for instance FMOC-Teg (i.e., Fmoc-11-amino-3,6,9-undecanoyl moiety), to the resin-bound peptide. Also the very first unit (at the C-terminal end of the sequence) can be connected to Rink amide resin or to cysteamine resin by means of this general coupling method described above; in the case of cysteamine resin the initial treatment with piperidine (steps 3 to 11) is not necessary at the first cycle. When a lactam-bridged cyclic compound was needed, the first cycle was carried out with 0.25 mmol of activated reagent in step 12 above (instead of usual 0.75 mmol) followed by resin capping between steps 12 and 13 by means of acetylation for 30 min using reagent mixture: 2 ml of acetic anhydride and 1 ml of 2,4,6-trimethylpyridine mixed in 4 ml of DMF.

When N-terminally acetylated product was needed the procedure above was carried out with the exception of acetic anhydride instead of the activated FMOC-amino acid at step 12 using reagent mixture: 2 ml of acetic anhydride and 1 ml of 2,4,6-trimethylpyridine mixed in 4 ml of DMF. Then the resin was washed with DMF and DCM before the cleavage process described below.

When lactam-bridged cyclic compounds were needed (lactam bridge between the N-terminus of D-Ala and side chain of γ-2-phenyl-isopropylester protected Glu) the procedure above was carried out with the exception of additional deprotection treatment (removal of the O-2-Ph-i-Pr protection of the side chain of Glu) of the resin after step 10 by shaking with four portions of 2% TFA (0.2 ml) solution in DCM (9.7 ml) containing 1% of triisopropylsilane (0.1 ml), each for two minutes followed by filtration. Then the resin was washed with 0.2 M DIPEA in DMF followed by washing steps 8-11. Next the resin was shaken for 5 hours with 0.75 mmol of the activation reagent PyAOP (i.e. 7-azabenzotriazol-1-yloxytris(pyrrolidino)phosphoniumhexafluorophosphate, Applied Biosystems, CAS No. 156311-83-0, Cat. No. GEN076533, molecular weight 521.4 g/mol) in 5 ml of DMF containing 1.5 mmol of DIPEA followed by final washings according to steps 13-18. Therefore this coupling was intramolecular without any FMOC-amino acid added in step 12. Then the resin was washed with DMF and DCM before the cleavage process described below.

After removal of the protecting FMOC group of the last amino acid of the peptide via steps 1. to 10., and acetylation according to the process described above or lactam bridge formation according to the process described above, the resin was washed with DCM, dried at argon flow and treated with three portions of the cleavage reagent mixture described below (each about 10 ml), each for one hour. The treatments were carried out under argon atmosphere in the way described above. After three hours from the beginning of the treatment the TFA solutions obtained by filtration were concentrated under reduced pressure using a rotary evaporator and were recharged with argon.

Cleavage from the resin was carried out using the following reagent mixture:

trifluoroacetic acid (TFA) 92.5 vol-%;

water 5.0 vol-%;

ethanedithiol 2.5 vol-%.

The cleavage mixture described above also simultaneously removed the following protecting groups: Tert-butoxycarbonyl (Boc) as used for protection of side chain of tryptophan, tert-butyl ester (OtBu)/(tBu) as used for protection of side chain carboxyl group or glutamic acid of hydroxyl group of tyrosine, trityl (Trt) as used for protection of side chain of cysteine.

When cystine bridged cyclic compounds were needed (disulfide bond between the side chains of cysteine amino acids), the crude peptide, after releasing from the resin according to the protocol described above, was dissolved in 0.05 M ammonium acetate solution at peptide concentration 0.01 M and it was allowed to stand at room temperature over night (air oxidation).

Purification was performed by reversed phase high-performance liquid chromatographic (HPLC) method using a “Waters 600” pump apparatus with a C-18 type column of particle size 10 micrometers, and a linear eluent gradient whose composition was changed during 30 minutes from 99.9% water/0.1% TFA to 99.9% acetonitrile/0.1% TFA; in some instances (indicated below) the eluent was buffered by 0.05 M ammonium acetate instead of 0.1% TFA. The dimensions of the HPLC columns were 25 cm×21.2 mm (Supelco cat. no. 567212-U) and 15 cm×10 mm (Supelco cat. no. 567208-U). Detection was based on absorbance at 218 nm and was carried out using a “Waters 2487” instrument. The fraction indicated by right mass spectrum was collected as product; to enhance purification the eluent composition limits were adjusted to achieve applicable gradient.

Compounds synthesized this way are constructed from “right to left” in the conventionally (also in this text) presented sequence, i.e. starting from the C-terminal end of the peptide chain.

Mass Spectral Method Employed:

Matrix Assisted Laser Desorption Ionization—Time of Flight (MALDI-TOF).

Type of the Instrument:

Bruker Ultraflex MALDI TOF/TOF mass spectrometer.

Supplier of the Instrument:

Bruker Daltonik GmbH,

Bremen,

Germany.

Maldi-Tof Positive Ion Reflector Mode:

External standards: Angiotensin II, angiotensin II, substance P, bombesin, ACTH(1-17) ACTH(18-39), somatostatin 28 and bradykinin 1-7.

Matrix: alpha-cyano-4-hydroxycinnamic acid (2 mg/ml solution in aqueous 60% acetonitrile containing 0.1% of trifluoroacetic acid, or acetone only for acid sensitive samples).

Maldi-Tof Negative Ion Reflector Mode:

External standards: cholecystokinin and glucagon or [Glu1]-fibrinogen peptide B.

Matrix: alpha-cyano-4-hydroxycinnamic acid (saturated solution in acetone).

Sample Preparation:

The specimen was mixed at a 10-100 picomol/microliter concentration with the matrix solution as described and dried onto the target.

Ionization by “shooting” in vacuo by nitrogen laser at wavelength 337 nm. The Voltage of the probe plate was 19 kV in positive ion reflector mode and −19 kV in the negative ion reflector mode.

General Remarks About the Spectra (Concerning Positive Ion Mode Only):

In all cases the M+1 (i.e., the one proton adduct) signal with its typical fine structure based on isotope satellites was clearly predominant. In almost all cases, the M+1 signal pattern was accompanied by a similar but markedly weaker band of peaks at M+23 (Na⁺ adduct). In addition to the bands at M+1 and M+23, also bands at M+39 (K⁺ adduct) or M+56 (Fe⁺ adduct) could be observed in many cases.

The molecular mass values reported within synthesis examples correspond to the most abundant isotopes of each element, i.e., the ‘exact masses’.

Synthesis of Targeting Agent HP202

The synthesis of ADGA-CYGFVWGEC-Teg3-amide (SEQ ID NO: 34), i.e. Ala-Asp-Gly-Ala-Cys-Tyr-Gly-Phe-Val-Trp-Gly-Glu-Cys-Teg-Teg-Teg-NH₂ (SEQ ID NO:34), that has solubility-enhancing units and is cyclic by virtue of the disulfide bond between the two cysteines (Cys), was carried out using an Applied Biosystems 433A peptide synthesis instrument and based on Rink amide MBHA Resin and solid phase Fmoc-chemistry and regular protected amino acid reagents (including Fmoc-11-amino-3,6,9-undecanoic acid i.e. Fmoc-Teg-OH that was used in the regular manner). After release from the resin, the crude peptide was cyclized according to the air oxidation protocol described above and purified by RP-HPLC.

The identification of the product was based on MALDI-TOF mass spectrum: Observed positive ion M+1: 1943.81 Da; M+Na: 1965.81 Da.

Calculated isotopic M: 1942.83.

Synthesis of Targeting Agent HP203

The synthesis of ADGA-CYGFLWGQC-Teg3-amide (SEQ ID NO:35), i.e. Ala-Asp-Gly-Ala-Cys-Tyr-Gly-Phe-Leu-Trp-Gly-Gln-Cys-Teg-Teg-Teg-NH₂ (SEQ ID NO: 35), that has solubility-enhancing units and is cyclic by virtue of disulfide bond between the two cysteines (Cys), was carried out using an Applied Biosystems 433A peptide synthesis instrument and based on Rink amide MBHA Resin and solid phase Fmoc-chemistry and regular protected amino acid reagents (including Fmoc-11-amino-3,6,9-undecanoic acid i.e. Fmoc-Teg-OH that was used in the regular manner). After release from the resin, the crude peptide was cyclized according to the air oxidation protocol described above and purified by RP-HPLC.

The identification of the product was based on MALDI-TOF mass spectrum: Observed positive ion M+Na: 1976.64 Da.

Calculated isotopic M: 1953.86.

Synthesis of Control Peptide HP204

The synthesis of ADGA-CWEGGLYFC-Teg3-amide (SEQ ID NO:36), i.e. Ala-Asp-Gly-Ala-Cys-Trp-Glu-Gly-Gly-Leu-Tyr-Phe-Cys-Teg-Teg-Teg-N H₂ (SEQ ID NO:36), that has a solubility-enhancing units at the C-terminus and is cyclic by virtue of disulfide bond between the two cysteines (Cys), was carried out using an Applied Biosystems 433A peptide synthesis instrument and based on Rink amide MBHA Resin and solid phase Fmoc-chemistry and regular protected amino acid reagents (including Fmoc-11-amino-3,6,9-undecanoic acid i.e. Fmoc-Teg-OH that was used in the regular manner). After release from the resin, the crude peptide was cyclized according to the air oxidation protocol described above and purified by RP-HPLC.

The identification of the product was based on MALDI-TOF mass spectrum: Observed positive ion M+Na: 1979.7 Da.

Calculated isotopic M: 1956.8.

Synthesis of Targeting Agent KK12

The synthesis of Ac-CYGFVWGEC-(TEGC-Glu)2-NHCH₂CH₂NH₂ (SEQ ID NO: 9) i.e. Acetyl-Cys-Tyr-Gly-Phe-Val-Trp-Gly-Glu-Cys-(TEGC-Glu)₂-NH—CH₂CH₂—NH₂ (SEQ ID NO:9), cyclic by virtue of a cystine bridge between the two cysteines (Cys), and comprising solubility-enhancing units, was carried out manually according to the general protocol described above, based on 1,2-Diaminoethanetrityl resin and solid phase Fmoc-chemistry and regular protected amino acid reagents (including Fmoc-TEGC-OH that was used in the regular manner). The N-terminus was acetylated according to the general protocol described above and the compound was cleaved off the resin, as described above, and cyclized in 0.05 M ammonium bicarbonate solution exposed by air at room temperature over night, and then purified by RP-HPLC.

The identification of the product was based on MALDI-TOF mass spectrum: Observed positive ion M+1: 1809.85 Da, M+Na: 1831.68 Da, M+K:1847.65 Da.

Calculated isotopic M: 1808.8 Da.

Synthesis of Europium-Labeled Targeting Agent MJ069

The synthesis of cystine-bridged targeting agent Acetyl-CYGFVWGEC-(TEGC-Glu)₂-NHCH2CH2NHC(S)-p-NH-Benzyl-DTPA-Eu (SEQ ID NO: 9) comprising a europium chelating effector unit, a solubility-enhancing spacer unit and a targeting unit CYGFVWGEC (SEQ ID NO: 9), i.e. Cys-Tyr-Gly-Phe-Val-Trp-Gly-Glu-Cys (SEQ ID NO: 9), cyclic by virtue of the disulfide bond between the cysteines (Cys), was performed as follows: 2.5 mol equivalents (0.00363 mmol) of ITC-DTPA (p-SCN-Benzyl-DTPA, Macrocyclics, Dallas Tex., MW: 649.92 g/mol) was dissolved in 0.33 ml of 0.05 M aqueous NaHCO₃. Then 1 molar equivalent (0.00145 mmol) of peptide KK22, described above, in 0.2 ml of aqueous NaHCO₃, was added to the ITC-DTPA solution and pH was adjusted to 9 with aqueous NaOH. After gentle stirring overnight, at room temperature and protected from light, 3 mol equivalents (0.00436 mmol) of EuCl₃×6H₂O, in 50 μL of H₂O, was added to the reaction mixture. Then, after gentle stirring for 4 h at room temperature, the mixture was dissolved in 100 μL of 1 M NaHCO₃.

The compound was purified by RP-HPLC at water-acetonitrile eluent gradient buffered by 0.05 M ammonium acetate, and the desired product was identified by MALDI-TOF mass spectrometry: Observed positive ions M+1: 2497.80 Da, M+Na: 2521.80 Da, M+K: 2537.77 Da.

Calculated isotopic M: 2498.814 Da.

Synthesis of Control Peptide HP238

The synthesis of a linear control peptide Ac-aWEYGVGFE-(TEGC-Glu)₂-EAT, i.e. Acetyl-(D-Ala)-Trp-Glu-Tyr-Gly-Val-Gly-Phe-Glu-(TEGC-Glu)₂-EAT comprising a sulfhydryl bearing linker unit via a solubility-enhancing spacer unit at the C-terminus of the peptide sequence, was carried out manually according to the general protocol described above and was based on cysteamine-2-chlorotrityl resin and solid phase Fmoc-chemistry and regular protected amino acid reagents (including unusual Fmoc-TEGC-OH that was used in the regular manner). The N-terminus was acetylated according to the general protocol described above and after release from the resin the crude peptide was purified by RP-HPLC.

The identification of the product was based on MALDI-TOF mass spectrum: Observed negative ion M−1: 1820.44 Da.

Calculated isotopic M: 1821.8 Da.

Synthesis of Targeting Agent MJ012

The synthesis of linear targeting agent Ac-aYGFVWGEE-(Teg-Glu)₂-EAT (SEQ ID NO: 17), i.e. Acetyl-(D-Ala)-Tyr-Gly-Phe-Val-Trp-Gly-Glu-Glu-(Teg-Glu)₂-EAT (SEQ ID NO: 17) comprising targeting unit aYGFVWGEE (SEQ ID NO: 17) and sulfhydryl bearing linker agent via solubility-enhancing spacer units at the C-terminus of the targeting unit was carried out manually according to the general protocol described above and was based on cysteamine-2-chlorotrityl resin and solid phase Fmoc-chemistry and regular protected amino acid reagents (including Fmoc-Teg-OH that was used in the regular manner). The N-terminus was acetylated according to the general protocol described above and after release from the resin the crude peptide was purified by RP-HPLC.

The identification of the product was based on MALDI-TOF mass spectrum: Observed positive ion M+Na: 1816.77 Da; M+K: 1832.75.

Calculated isotopic M: 1793.77Da.

Synthesis of Targeting Agent MJ080

The synthesis of lactam-bridged targeting agent a*YGFVWGEE*-Teg-Glu (SEQ ID NO: 17), i.e. (D-Ala)*-Tyr-Gly-Phe-Val-Trp-Gly-Glu-Glu*-Teg-Glu (SEQ ID NO: 17), cyclic by virtue of a lactam bridge between the N-terminus and the side-chain of E* (glutamic acid) and bearing a solubility-enhancing unit at the C-terminus of the targeting unit, was carried out manually according to the general protocol described above and was based on Fmoc-Glu(OtBu)-Wang resin and solid phase Fmoc-chemistry and regular protected amino acid reagents (including Fmoc-Teg-OH that was used in the regular manner). The lactam bridge was prepared according to the general protocol described above and the peptide was purified by RP-HPLC.

The identification of the product was based on MALDI-TOF mass spectrum: Observed positive ion M+Na: 1371.60 Da, M+Na: 1396.59 Da, M+K: 1409.56 Da.

Calculated isotopic M: 1370.60 Da.

Synthesis of Targeting Agent MJ013

The synthesis of lactam-bridged targeting agent a*YGFVWGEE*-(Teg-Glu)2-EAT (SEQ ID NO: 17), i.e. (D-Ala)*-Tyr-Gly-Phe-Val-Trp-Gly-Glu-Glu*-(Teg-Glu)₂-EAT (SEQ ID NO: 17) (lactam bridge between N-terminus of a* and side chain COOH group of E*) comprising the targeting unit a*YGFVWGEE* and a sulfhydryl bearing linker agent via solubility-enhancing spacer units at the C-terminus of the targeting unit, was carried out manually according to the general protocol described above and was based on cysteamine-2-chlorotrityl resin and solid phase Fmoc-chemistry and regular protected amino acid reagents (including Fmoc-Teg-OH that was used in the regular manner). The lactam bridge was prepared according to the general protocol described above and the peptide was purified by RP-HPLC.

The identification of the product was based on MALDI-TOF mass spectrum: Observed positive ion M+Na: 1756,73 Da; M+K: 1772,71 Da.

Calculated isotopic M: 1733,75 Da.

Preparation of Europium Labeling Agent Eu-DTPA-IAA

The Synthesis of Eu-DTPA-IAA i.e. Eu³⁺-chelate of (p-iodoacetamidobenzyl)diethylenetriamine-N,N,N′,N″,N″-pentaacetic acid was performed in three steps. The synthesis was started from (t-BuO)5DTPA-Bz-NH₂ purchased from Macrocyclics, Dallas, Tex. (0.300 mmol), which was allowed to react with iodoacetic anhydride (0.330 mmol) and triethylamine (0.315 mmol) in dichloromethane (6 ml) at room temperature for 2 hours. The crude product was purified by flash chromatography in silica gel column eluted by 30% ethyl acetate in hexane. Then the flash-purified t-BuO-protected DTPA-IM was deprotected by excess of neat TFA at room temperature over night and, without purification, labeled with europium (2 mol equivalent of aqueous EuCl₃×H₂O) in ammonium acetate buffer (3 mol equivalent) at room temperature over night. This final Eu-DTPA-IAA compound was purified by RP-HPLC at water-acetonitrile eluent gradient buffered by 0.05 M ammonium acetate and the desired product was identified by MALDI-TOF mass spectrometry. Calculated M: 816.00 Da; obtained positive ion M+H: 816.96 Da.

Synthesis of Europium-Labeled Targeting Agent MJ017

The synthesis of linear targeting agent Ac-aYGFVWGEE-(Teg-Glu)₂-AMB-DTPA-Eu (SEQ ID NO: 17), i.e. Acetyl-D-Ala-Tyr-Gly-Phe-Val-Trp-Gly-Glu-Glu-(Teg-Glu)₂-NHCH₂CH₂—S—CH₂C(O)-p-aminobenzyl-DTPA-Eu (SEQ ID NO: 17) comprising targeting unit aYGFVWGEE (SEQ ID NO: 17) and a europium-bearing DTPA chelate coupled via a thioether bond (included in ‘AMB’ linkage) and solubility-enhancing spacer units at the C-terminus of the targeting unit, was carried out in aq Na—HCO₃.at pH 8.5. The peptide (MJ012, 1 eq) was dissolved in 0.05 M NaHCO₃ and the Eu³⁺-chelate of (p-iodoacetamidobenzyl)diethylenetriamine-N,N,N′,N″,N″-penta-acetic acid (Eu-DTPA-IM, 2 eq) in 0.05 NaHCO₃ was added to the peptide solution. After this, pH was adjusted to 8.5, the solution was protected from light and allowed to stay overnight at 37° C. The DTPA-Eu labeled peptide was purified by RP-HPLC at water-acetonitrile eluent gradient buffered by 0.05 M ammonium acetate.

The identification of the product was based on MALDI-TOF mass spectrum: Observed negative ion M−1: 2480.68 Da.

Calculated isotopic M: 2481.86 Da.

Synthesis of Europium-Labeled Targeting Agent MJ018

The synthesis of lactam-bridged targeting agent a*YGFVWGEE*-(Teg-E)₂-AMB-DTPA-Eu (SEQ ID NO: 17), i.e. D-Ala*-Tyr-Gly-Phe-Val-Trp-Gly-Glu-Glu*-(Teg-Glu)₂-NHCH₂CH₂—S—CH₂C(O)-p-aminobenzyl-DTPA-Eu (SEQ ID NO: 17) (lactam bridge between N-terminus of a* and the side chain COOH group of E*) comprising targeting unit a*YGFVWGEE* (SEQ ID NO: 17) and a europium-bearing DTPA chelate coupled via a thioether bond (included in ‘AMB’ linkage) and solubility-enhancing spacer units at the C-terminus of the targeting unit, was carried out in aq Na—HCO₃.at pH 8.5. The peptide (MJ013, 1 eq) was dissolved in 0.05 M NaHCO₃ and the Eu³⁺-chelate of (p-iodoacetamidobenzyl)diethylenetriamine-N,N,N′,N″,N″-penta-acetic acid (Eu-DTPA-IM, 2-3 eq) in 0.05 NaHCO₃ was added to the peptide solution. After pH was adjusted to 8.5, the solution was protected from light and allowed to stay overnight at 37° C. The DTPA-Eu labeled peptide was purified by RP-HPLC at water-acetonitrile eluent gradient buffered by 0.05 M ammonium acetate.

The identification of the product was based on MALDI-TOF mass spectrum: Observed negative ion M−1: 2420.67 Da.

Calculated isotopic M: 2421.84 Da.

### Example 3

Selective Binding of Colorectal Cancer Cells to Immobilized Targeting Agents

In these examples the following cell lines and culture conditions were used, where not otherwise indicated:

The human colorectal cancer HCT-15 cell line (ATCC: CCL-225), called herein also “HCT-15”, was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

The human colon adenocarcinoma cell line LoVo (ATCC:CCL-229), called herein “LoVo”, was cultured in Ham's F-12 medium adjusted to contain 2 mM L-glutamine, 1% penicillin/streptomycin, 1.5 g/L sodium bicarbonate and 10% fetal bovine serum.

The colorectal cancer cell line HCT-15-LM1 was developed as follows. The cell culture was started with cancer cells from lung metastases which had developed after injection of human colorectal cancer HCT-15 cells into the bloodstream of a mouse. The inoculation procedure was then repeated with HCT-15-LM1 cells leading to the establishment of another metastatic cell line, HCT-15-LM2.

The mouse fibroblast line NIH3T3, called herein also “NIH3T3”, has been described previously by Koga et al. in Gann, 1979, 70: 585-591. The cell line was cultured in DMEM medium adjusted to contain 2 mM L-glutamine, 1% penicillin/streptomycin, and 10% fetal bovine serum.

The mouse vascular endothelial cell line SVEC4-10, called herein also “SVEC4-10”, has been described previously by O'Connell et al. in J. Immunol., 1990, 144: 521-525. The cell line was cultured in DMEM medium adjusted to contain 2 mM L-glutamine, 1% penicillin/streptomycin, and 10% fetal bovine serum.

The human melanoma cell line C8161 has been described previously by Welch et al. in Int. J. Cancer, 1991, 47: 227-237. A more metastatic cell line C8161T, called herein also “C8161T”, was developed by culturing cells from a subcutaneous melanoma tumor developed after inoculation of C8161 melanoma cells on the flank of a nude mouse. The cell line was cultured in DMEM medium adjusted to contain 2 mM L-glutamine, 1% penicillin/streptomycin, and 10% fetal bovine serum.

The human oral squamous cell carcinoma line HSC-3, called herein “HSC-3” (JCRB Cell Bank 0623, National Institute of Health Sciences, Japan) was cultivated in 1:1 DMEM and Ham's F 12 medium containing 10% FBS, 1% penicillin/streptomycin, L-glutamate and sodium pyruvate and 0.4 ng/ml hydrocortisone.

Preparation of plates for assays. Wells of Reacti-Bind Maleimide activated clear strip plate (Pierce, Prod#. 15150) were coated with targeting agents of this invention at a concentration of 30 μg/ml. The incubation was carried out of overnight at 20° C. The binding buffer containing unbound peptide was removed from the wells.

The wells were blocked with blocking buffer (0.5% BSA, 0.05% Tween20 in phosphate buffer saline (PBS). PBS was prepared as follows: 40 g of NaCl, 1 g of KCl, 7 g of Na₂HPO₄×2H₂O and 1 g of KH₂PO₄ were dissolved in 1000 ml of deionized H₂O. Blank wells as controls were prepared by treating empty wells with blocking buffer. The plates were incubated 1 hour 30 min at 20° C. After incubation the plate was washed three times with PBS, pH 7.4.

Cell binding assay. 75000 cells in volume of 150 μl of medium were added into coated wells and were incubated for 30 minutes at 37° C. After cell binding, the wells were washed with PBS for 30 minutes. Detection of targeting agent bound cells was based on the MTT assay (described in detail in Example 6, Cytotoxicity). 10 μl of MTT reagent and 90 μl of medium were added to the wells. The plate was incubated for three hours at +37° C. After the incubation, 100 μl of lysis buffer was added to the wells and let to incubated o/n 37° C. On following day the absorbance of plate was measured at 560 nm with ELISA-reader (ThermoLabsystems, Multiskan EX).

The colon cancer cell lines, HCT-15 and HCT-15-LM1, HSC-3 oral cancer cell line and C8161T melanoma cell line and control cell lines NIH-3T3 and SVEC4-10 and targeting agents MJ012 and MJ013 (described in Example 2) were used in the cell binding assays.

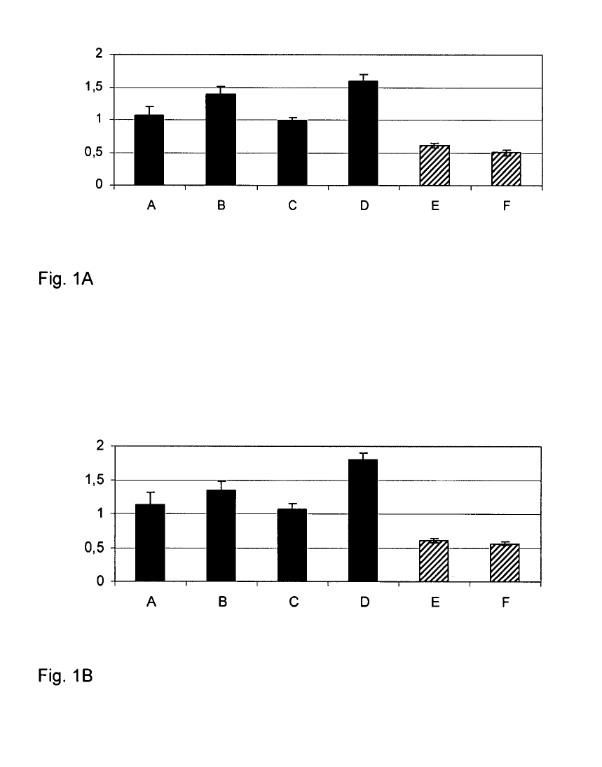

The results of the cell binding assay showing the selective binding of cancer cell lines to the targeting agents are shown in FIG. 1. The colon cancer cell lines HCT-15 (A), HCT-15-LM1 (B), tongue carcinoma HSC-3 (C) and melanoma cell line C8161T (D), bind selectively to the immobilized targeting agents MJ012 (FIG. 1A) and MJ013 (FIG. 1B), whereas the control cell lines, mouse fibroblast cell line NIH3T3 (E) and murine endothelial cell line SVEC4-10 (F) show significantly less binding. The results are shown as measured absorbance at 560 nm.

### Example 4

Accumulation of Targeting CYGFLWGQC-Phage (SEQ ID NO: 11) in Primary Tumors and Lung Metastases in Mice

In this example the biodistribution of the targeting phage displaying peptide sequence CYGFLWGQC (SEQ ID NO: 11) on the surface is shown for primary tumors of colon cancer and also for lung metastases in mice after iv. injection of HCT-15-LM1colon cancer cells and metastase development. It is shown that the tested targeting peptide displaying phage according to the present invention selectively targets to colon cancer tumors and lung metastases with high tumor/muscle ratio.

For production of experimental tumors 1×10⁶ cells of HCT-15-LM1 line (described in Example 3) were injected subcutaneously into both flanks of athymic-nu nude mice strain (Harlan Laboratories). The tumors from six mice were harvested when they had reached a weight of about 0.4 g.

To produce lung metastases, 6×10⁶ cells of HCT-15-LM1 cell line were injected into the bloodstream of two mice. The animals were weighed twice a week to monitor the weight loss indicating possible metastase development.

Tumor-bearing mice were anesthetized by 0.02 ml/g body weight of Avertin [10 g 2,2,2-tribromoethanol (Fluka) in 10 ml 2-methyl-2-butanol (Sigma Aldrich)] intraperitoneally (i.p.). To determine the accumulation of the targeting phage, 10⁹ transducing units of CYGFLWGQC-peptide (SEQ ID NO: 11) displaying phage were injected to the tail vein of the mouse. After 15 minutes circulation time the animals were perfused through heart with 20-60 ml DMEM. Tumors and control organs were removed and washed with DMEM+PI before they were homogenized. After this the samples were washed three times with DMEM-PI containing 1% BSA (bovine serum albumin).

0.5 ml K91kan bacteria, OD600 (optical density of 600 nm) 1-1.5, in LB (Lurian broth) containing 100ug/ml kanamycin (kan) were infected with the pellet containing phage particles binding to the tissues. After infection volume was increased to 5 ml with LB containing 100 ug/ml kan. 100, 10, 1 and 0.1 μl from the solution of infected bacteria were plated on LB agar plates containing 40 μg/ml tetracycline (tet). The plates were incubated overnight at +37° C. Next day the colonies were counted and the results were analyzed.

The number of colonies/1 g tissue showed the following tumor versus muscle phage binding ratios. n₁ is the number of tumors and n₂ is the number of mice.

| | | |
|---|---|---|
| Primary tumor: muscle | 23.8:1 | n₁ = 6 |
| Metastase: muscle | 39.4:1 | n₂ = 2 |

### Example 5

Targeting Peptide Biodistribution

In this example biodistribution of the targeting agents MJ017 and MJ018 (described in Example 2) is shown for primary tumors of HTC-15 and lung metastases of HCT-15-LM1. It is shown that the tested targeting agents according to the present invention selectively target to primary tumors in vivo as well as lung metastases but not to normal tissues or organs.

For production of experimental tumors 22.5×10⁶ cells of HCT-15 line (described in Example 3) were injected subcutaneously into both flanks of athymic-nu nude mice strain (Harlan Laboratories). Tumors were harvested when they had reached a weight of about 0.1 g. Tumor-bearing mice were anesthetized by 0.02 ml/g body weight of Avertin [10 g 2,2,2-tribromoethanol (Fluka) in 10 ml 2-methyl-2-butanol (Sigma Aldrich)] intraperitoneally (i.p.).

To determine the biodistribution pattern of the targeting agents MJ017 and MJ018, 4 nmol of MJ017-Eu and MJ018-Eu targeting agent was injected into the tail vein of athymic nude mice in a volume of 200 μl in physiological saline solution (Baxter). Targeting agent was allowed to circulate for 15 min. Mice were then perfused through the heart with 60 ml of physiological saline. Organs and tissues, including tumors were collected and weighed.

For determination of the Eu content of the tumors and control organs, 0.1-0.2 g of tissue was transferred into lysis buffer (20 mM Tris-HCl, 150 mM NaCl, 1% Nonident P40, pH 7.5) and homogenized. Eu-content was analyzed from tissue lysates prepared into the Enhancement solution (Perkin Elmer Wallac Ltd., Turku, Finland) and transferred to DELFIA Micro titration strip plates (Perkin Elmer Wallac Ltd, Turku, Finland). Time-resolved fluorescence was measured after incubation with a Wallac 1420 VICTOR³™ V plate reader using a D615 nm filter.

The comparison of the amount of europium detected in the mouse tissues showed that the MJ017-Eu and MJ018-Eu targeting agents accumulated strongly and selectively in HTC-15 tumors and HCT-15-LM1 lung metastases compared to normal tissue, except for the kidney showing high signal due to excretion of the agent via these routes.

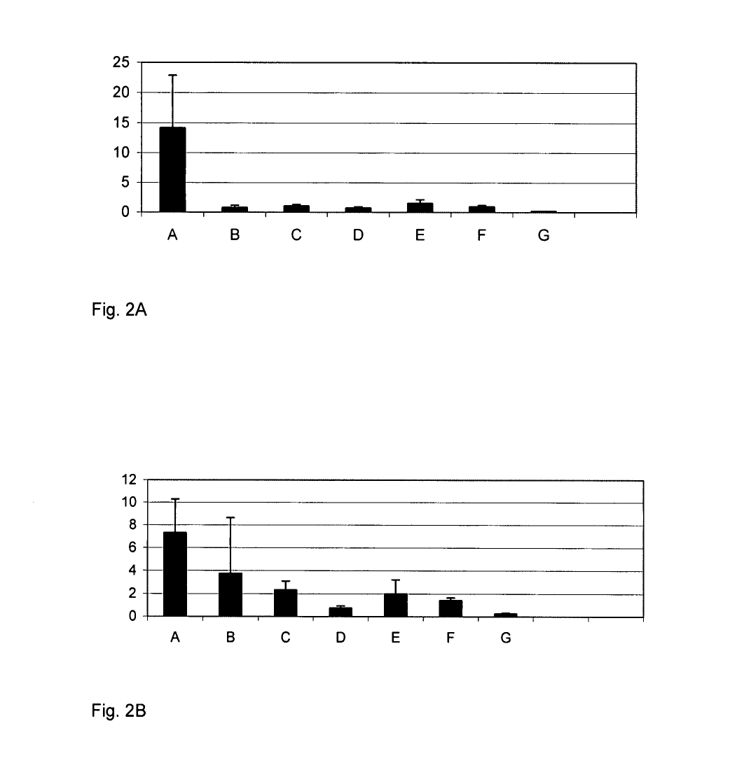

The observed high tumor-to-muscle ratio further proves the highly selective binding of MJ017-Eu and MJ018-Eu to HTC-15 tumors shown in FIG. 2. A=tumor, B=heart, C=lung, D=Iiver, E=spleen, F=small intestine and G=brain.

The ratio of lung metastase Eu-content to-muscle Eu-content of targeting agent MJ017 was 4.1.

Thus, the used targeting agent shows highly selective tumor and metastase targeting properties.

### Example 6

Cytotoxicity Assay with CYGFLWGQC-Peptide (SEQ ID NO: 11)

In this assay LoVo cell line (described in example 3) was exposed to two different concentrations (5 μg/ml and 138 μg/ml) of targeting unit HP203 (described in Example 2) for three days to test the toxicity of the peptides. The measurement of cell viability was done with MTT (Thiazolyl blue, Sigma M-5655) tetrazolium salt. MTT is cleaved to water-insoluble formazan dye by the “succinate-tetrazolium reductase” system, which is active only in viable cells. After formazan was solubilized by 10% SDS-0.01 M HCl, it was quantified in an ELISA spectrometer (ThermoLabsystems Multiscan EX) at 560 nm. CuSAO₂ [trans-bis(salicylaldoximato)copper(II)] 7.5 μg/ml was used as a positive control for 100% toxicity.

Procedure. Cells were trypsinized from the cell culture dish (ø9 cm) with 1 ml of TE for 1-5 minutes and moved to a 50 ml Falcon tube. After this the volume was increased to 20 ml of cell line specific medium and cells were transferred to a Bürker chamber and diluted in medium to a concentration of 2500-3500 cells/100 μl depending on cell line. Two or three 96-well micro plates, 24 h, 48 h and 72 h were prepared as follows: the first column of the 96-well plate was filled with 100 μl medium/well (w/o cells), and the rest of the columns needed for the experiment with 100 μl of the cell solution so that each well contains 2500-3000 cells. After this the cells were let to attach over night in a cell culture incubator.

Next day 40 μl of medium was removed from all wells except from the ones with only medium and one column with cells. Then 40 μl of HP203 targeting unit in appropriate medium were added to the wells in two concentrations, so that final concentrations were 5 μg/ml and 138 μg/ml, and the volume of the wells was raised back to 100 μl. Similarly, 40 μl of reference substance Cu(SAO)₂ were added to all the wells in one column so that the final concentration was 7.5 μg/ml. The plates were incubated in an incubator for 24 h, 48 h or 72 h. The next day the cell morphology was analyzed with a microscope. After this 10 μl of MTT reagent 5 mg/ml in PBS were added to all wells on the plate and the plate was incubated for 3 h at 37° C. Finally, 100 μl of 10% SDS in 0.01M HCl were added to all the wells and the microplate was incubated over night at 37° C.

The next day, the MTT assay described above was performed. The viable count (v.c.) was calculated as:

$$\frac{\begin{matrix}Average toxicated cell absorbance - \\ Average DMEM absorbance\end{matrix}}{\begin{matrix}Average living cell absorbance - \\ Average DMEM absorbance\end{matrix}} = Viable count$$

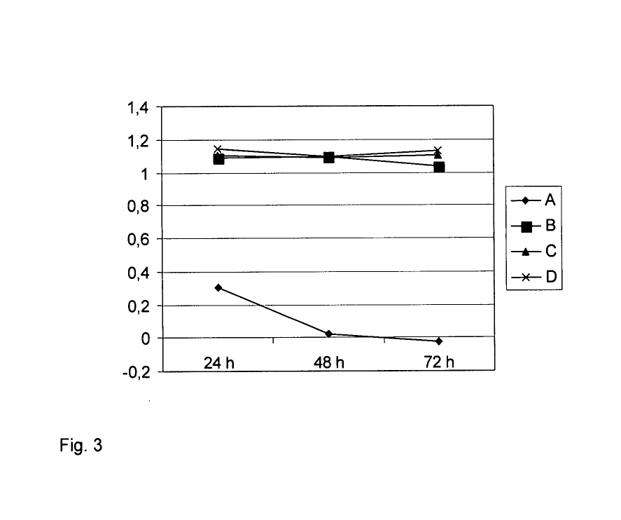

HP203 targeting unit was found non-toxic for the tested cell line whereas CuSAO₂ 7.5 μg/ml, used as a positive control, showed 100% cell killing. An example of the results is shown as viable count vs. time in FIG. 3 where A=Cu(SAO)₂, B=DMSO, C=138 μg/ml HP203 and D=5 μg/ml HP203.

### Example 7

In Vivo Toxicity Study

1 mg of targeting unit HP203 (described in Example 2) was injected i.v. into the tail vein of three Athymic nude mice in a volume of 100 μl of sterile physiological saline. The behavior of mice was observed during 30 min right after injection and during 15 min on the following day (comparison to non-injected mouse). Injection of targeting unit HP203 did not have any toxic effect on mice.

### Example 8

In Vivo Tumor Reduction

This example is provided to show that targeting units MJ017 and MJ018 (described in Example 2) when coupled to cytotoxic substances can be used as therapeutic agents for the treatment of colon cancer.

Production of experimental colon cancer tumors is described in Example 3. Mice bearing colon cancer tumors on their flanks are divided into two groups: Control animals receiving a standard dose of a cytotoxic substance administrated intravenously, and the test animals receiving an equal amount of said cytotoxic substance coupled to targeting unit MJ017 or MJ018. Body weight loss, indicating toxicity and tumor growth delay indicating antitumor activity are followed. Weight and tumor size are measured every third day for four weeks. The experiment is terminated before the final endpoint if the body weight of the animal is reduced by 30% from the baseline.

Net body weight loss is calculated as:

$$\frac{initial weight - lowest weight}{initial weight} \times 100 \%$$

Mean tumor volume (mm³) is calculated according the formula with measurements using calipers:
(length×height×width×π)/6

By comparing the results between the mice in the test and the control groups it can be shown that targeting units MJ017 and MJ018 coupled to cytotoxic substance can reduce body weight loss and tumor growth in mice and thus show therapeutic activity.

1. A targeting unit having a peptide sequence:

| | |
|---|---|
| C_{y}-Y-G-F-X-W-G-Z-C_{yy} | (SEQ ID NO: 25) |

or a pharmaceutically or physiologically or diagnostically acceptable salt thereof, wherein, Y is tyrosine or a structural or functional analogue thereof; G is glycine or a structural or functional analogue thereof; F is phenylalanine or a structural or functional analogue thereof; X is alanine, valine, leucine, or isoleucine or a structural or functional analogue thereof; W is tryptophan or a structural or functional analogue thereof; Z is glutamine or glutamic acid, or a structural or functional analogue thereof; and C_{y} and C_{yy} are optional entities forming a cyclic structure; said unit selectively targeting tumors.

2. The targeting unit according to claim 1, wherein said tumor is a colon cancer tumor.

3. The targeting unit according to claim 2, wherein said tumor is a metastasis originating from the colon.

4. The targeting unit according to claim 3, wherein said tumor is a lung metastasis.

5. The targeting unit according to claim 4, wherein the peptide is linear.

6. The targeting unit according to claim 5 selected from the group consisting of YGFVWGE (SEQ ID NO. 1), YGFVWGQ (SEQ ID NO. 2), YGFLWGQ (SEQ ID NO. 3), YGFLWGE (SEQ ID NO. 4), YGFAWGQ (SEQ ID NO. 5), YGFAWGE (SEQ ID NO. 6), YGFIWGQ (SEQ ID NO. 7), YGFIWGE (SEQ ID NO. 8), aYGFVWGEE (SEQ ID NO.17), aYGFVWGQE (SEQ ID NO.18), aYGFLWGQE (SEQ ID NO.19), aYGFLWGEE (SEQ ID NO. 20), aYGFAW-GQE (SEQ ID NO. 21), aYGFAWGEE (SEQ ID NO. 22), aYGFIWGQE (SEQ ID NO. 23) and aYGFIWGEE (SEQ ID NO. 24).

7. The targeting unit according to 4, wherein the peptide is cyclic or forms part of a cyclic structure.

8. The targeting unit according to claim 7, wherein the cyclic structure is a lactam.

9. The targeting unit according to claim 8, wherein C_{y} is selected from the group consisting of glutamic acid, aspartic acid and structural or functional analogues thereof when C_{yy} is selected from the group consisting of lysine, ornithine and structural or functional analogues thereof; or C_{y} is selected from the group consisting of lysine, ornithine, structural or functional analogues thereof and an N-terminal D-amino acid when C_{yy} is selected from the group consisting of glutamic acid, aspartic acid and structural or functional analogues thereof.

10. The targeting unit according to claim 9 wherein C_{y} is D-alanine and C_{yy} is glutamic acid.

11. The targeting unit according to claim 10 selected from the group consisting of aYGFVWGEE (SEQ ID NO. 17), aYGFVWGQE (SEQ ID NO. 18), aYGFLWGQE (SEQ ID NO. 19), aYGFLWGEE (SEQ ID NO. 20), aYGFAWGQE (SEQ ID NO. 21), aYGFAWGEE (SEQ ID NO. 22), aYGFIWGQE (SEQ ID NO. 23) and aYGFIWGEE (SEQ ID NO. 24).

12. The targeting unit according to claim 7, wherein the cyclic structure is formed through a disulphide bond.

13. The targeting unit according to claim 12, wherein C_{y} and C_{yy} are cysteine or a structural or functional analogue thereof.

14. The targeting unit according to claim 13 selected from the group consisting of CYGFVWGEC (SEQ ID NO. 9), CYGFVWGQC (SEQ ID NO. 10), CYGFLWGQC (SEQ ID NO. 11), CYGFLWGEC (SEQ ID NO. 12), CYGFAWGQC (SEQ ID NO. 13), CYGFAWGEC (SEQ ID NO. 14), CYGFIWGQC (SEQ ID NO. 15) and CYGFIWGEC (SEQ ID NO. 16).

15. A tumor targeting agent comprising at least one targeting unit of claim 1, directly or indirectly coupled to at least one effector unit.

16. The tumor targeting agent according to claim 15, wherein the effector unit is a therapeutic substance, a directly or indirectly detectable substance, or a substance having binding ability.

17. The tumor targeting agent according to claim 16, wherein the detectable substance comprises a chelator, a complexed metal, an enriched isotope, radioactive material, a paramagnetic substance, an affinity label, a fluorescent label, a luminescent label, a PET-active substance or a SPECT-active substance.

18. The tumor targeting agent according to claim 16, wherein the therapeutic substance is selected from the group consisting of cytotoxic, cytostatic, immunomodulating and radiation emitting substances.

19. The tumor targeting agent according to claim 15, further comprising at least one optional unit.

20. The tumor targeting agent according to claim 19, wherein said optional unit is an aqueous-solubility enhancing unit.

21. The tumor targeting agent according to claim 20, wherein said aqueous-solubility enhancing unit comprises at least one unit according to Formula I:
—(CH₂)ₘ—O (I)
where m is an integer of value 1 to 4;
or at least one unit according to Formula II:
-(A)ₛ-Y (II)
where (A)s is a spacer group wherein each A is independently CR₁R₂,
each R₁ and R₂ is independently selected from the group of hydrogen, hydroxyl, C₁₋₃ alkyl and C₁₋₃ hydroxyalkyl,
s is an integer of value 0 to 5, and
Y is selected from the group of COOH, CONH₂, NH₂ and guanyl;
or at least one unit according to Formula III: 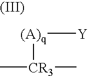
where (A)_{q} is a spacer group wherein each A is independently CR₁R₂,
q is an integer of value 1 to 5,
R₁, R₂ and R₃ are independently hydrogen, hydroxyl, C₁₋₃ alkyl or C₁₋₃ hydroxyalkyl, and
Y is selected from COOH, CONH₂, NH₂ and guanyl.

22. A diagnostic or pharmaceutical composition comprising at least one targeting unit according to claim 1.

23. Method of providing therapy comprising:
selectively targeting a tumor with a targeting unit having a peptide sequence:

| | |
|---|---|
| C_{y}-Y-G-F-X-W-G-Z-C_{yy} | (SEQ ID NO: 25) |

or a pharmaceutically or physiologically or diagnostically acceptable salt thereof, wherein, Y is tyrosine or a structural or functional analogue thereof; G is glycine or a structural or functional analogue thereof; F is phenylalanine or a structural or functional analogue thereof; X is alanine, valine, leucine, or isoleucine or a structural or functional analogue thereof; W is tryptophan or a structural or functional analogue thereof; Z is glutamine or glutamic acid, or a structural or functional analogue thereof; and C_{y} and C_{yy} are optional entities forming a cyclic structure.

24. Method of providing a diagnosis, comprising:
selectively targeting a tumor with a targeting unit having a peptide sequence:

| | |
|---|---|
| C_{y}-Y-G-F-X-W-G-Z-C_{yy} | (SEQ ID NO: 25) |

or a pharmaceutically or physiologically or diagnostically acceptable salt thereof, wherein, Y is tyrosine or a structural or functional analogue thereof; G is glycine or a structural or functional analogue thereof; F is phenylalanine or a structural or functional analogue thereof; X is alanine, valine, leucine, or isoleucine or a structural or functional analogue thereof; W is tryptophan or a structural or functional analogue thereof; Z is glutamine or glutamic acid, or a structural or functional analogue thereof; and C_{y} and C_{yy} are optional entities forming a cyclic structure.

25. Method of preparing a medicament for a cancer-related disease, comprising:
selectively targeting a tumor with a targeting unit having a peptide sequence:

| | |
|---|---|
| C_{y}-Y-G-F-X-W-G-Z-C_{yy} | (SEQ ID NO: 25) |

or a pharmaceutically or physiologically or diagnostically acceptable salt thereof, wherein, Y is tyrosine or a structural or functional analogue thereof; G is glycine or a structural or functional analogue thereof; F is phenylalanine or a structural or functional analogue thereof; X is alanine, valine, leucine, or isoleucine or a structural or functional analogue thereof; W is tryptophan or a structural or functional analogue thereof; Z is glutamine or glutamic acid, or a structural or functional analogue thereof; and C_{y} and C_{yy} are optional entities forming a cyclic structure.

26. Method according to claim 25, wherein said cancer related disease is a solid tumor or its metastases.

27. Method according to claim 26, wherein said solid tumor is selected from the group consisting of colon cancer, colorectal cancer and their lung metastases.

28. Method for the preparation of a diagnostic composition for the diagnosis of a cancer related disease, comprising:
selectively targeting a tumor with a targeting unit having a peptide sequence:

| | |
|---|---|
| C_{y}-Y-G-F-X-W-G-Z-C_{yy} | (SEQ ID NO: 25) |

or a pharmaceutically or physiologically or diagnostically acceptable salt thereof, wherein, Y is tyrosine or a structural or functional analogue thereof; G is glycine or a structural or functional analogue thereof; F is phenylalanine or a structural or functional analogue thereof; X is alanine, valine, leucine, or isoleucine or a structural or functional analogue thereof; W is tryptophan or a structural or functional analogue thereof; Z is glutamine or glutamic acid, or a structural or functional analogue thereof; and C_{y} and C_{yy} are optional entities forming a cyclic structure.

29. A method for treating a cancer related disease, comprising: providing to a subject in need thereof a therapeutically effective amount of a pharmaceutical composition according to claim 22.

30. The method according to claim 29, wherein said subject is a human.

31. The method according to claim 30, wherein said cancer related disease is selected from the group consisting of colon cancer, colorectal cancer and their metastases.

32. A method for diagnosis of cancer or cancer related diseases, comprising:
providing to a subject in need thereof a diagnostically suitable amount of a diagnostic composition according to claim 22.

33. The method according to claim 32, wherein said subject is a human.

34. The targeting unit according to claim 1, wherein the peptide is linear.

35. The targeting unit according to 1, wherein the peptide is cyclic or forms part of a cyclic structure.

36. A tumor targeting agent comprising at least one targeting unit of claim 6, directly or indirectly coupled to at least one effector unit.

37. A tumor targeting agent comprising at least one targeting unit of claim 11, directly or indirectly coupled to at least one effector unit.

38. A tumor targeting agent comprising at least one targeting unit of claim 14, directly or indirectly coupled to at least one effector unit.

39. The tumor targeting agent according to claim 18, further comprising at least one optional unit.

40. A diagnostic or pharmaceutical composition comprising at least one targeting agent according to claim 15.
